SEQUENCE LISTING (46 sequences)

SEQ ID NO: 1 - protein, 15 aa, unidentified
DFPFFVAIVFFLGRR

SEQ ID NO: 2 - protein, 20 aa, unidentified
HAFGTFTSDVSSYLEGQAAK

SEQ ID NO: 3 - protein, 13 aa, unidentified
APQTGIVDECCFR

SEQ ID NO: 4 - protein, 9 aa, unidentified
LEMYCAPLK

SEQ ID NO: 5 - protein, 9 aa, unidentified
GIVEECCFR

SEQ ID NO: 6 - protein, 16 aa, unidentified
SCDLALLETYCATPAK

SEQ ID NO: 7 - protein, 24 aa, unidentified
SAGCGCCPMCALPLGAACGVATAR

SEQ ID NO: 8 - protein, 8 aa, unidentified
CPPCTPER

SEQ ID NO: 9 - protein, 12 aa, unidentified
EPGCGCCSVCAR

SEQ ID NO: 10 - protein, 16 aa, unidentified
GDPECHLFYNEQQEAR

SEQ ID NO: 11 - protein, 16 aa, unidentified
GPLEHLYSLHIPNCDK

SEQ ID NO: 12 - protein, 23 aa, unidentified
LAACGPPPVAPPAAVAAVAGGAR

SEQ ID NO: 13 - protein, 12 aa, unidentified
LEGEACGVYTPR

SEQ ID NO: 14 - protein, 9 aa, unidentified
LIQGAPTIR

SEQ ID NO: 15 - protein, 13 aa, unidentified
TPCQQELDQVLER

SEQ ID NO: 16 - protein, 17 aa, unidentified
ALAQCAPPPAVCAELVR

SEQ ID NO: 17 - protein, 18 aa, unidentified
CQPSPDEARPLQALLDGR

SEQ ID NO: 18 - protein, 8 aa, unidentified
ETEYGPCR

SEQ ID NO: 19 - protein, 8 aa, unidentified
FLNVLSPR

SEQ ID NO: 20 - protein, 11 aa, unidentified
GLCVNASAVSR

SEQ ID NO: 21 - protein, 9 aa, unidentified
GVHIPNCDK

SEQ ID NO: 22 - protein, 15 aa, unidentified
SAGSVESPSVSSTHR

SEQ ID NO: 23 - protein, 11 aa, unidentified
YGQPLPGYTTK

SEQ ID NO: 24 - protein, 17 aa, unidentified
EDARPVPQGSCQSELHR

SEQ ID NO: 25 - protein, 14 aa, unidentified
GELDCHQLADSFRE

SEQ ID NO: 26 - protein, 8 aa, unidentified
LPGGLEPK

SEQ ID NO: 27 - protein, 15 aa, unidentified
THEDLYIIPIPNCDR

SEQ ID NO: 28 - protein, 9 aa, unidentified
AVYLPNCDR

SEQ ID NO: 29 - protein, 10 aa, unidentified
AVYLPNCDRK

SEQ ID NO: 30 - protein, 12 aa, unidentified
FVGGAENTAHPR

SEQ ID NO: 31 - protein, 10 aa, unidentified
APAVAEENPK

SEQ ID NO: 32 - protein, 22 aa, unidentified
EGQECGVYTPNCAPGLQCHPPK

SEQ ID NO: 33 - protein, 13 aa, unidentified
GAQTLYVPNCDHR

SEQ ID NO: 34 - protein, 15 aa, unidentified
HLDSVLQQLQTEVYR

SEQ ID NO: 35 - protein, 17 aa, unidentified
AGAAAGGPGVSGVCVCK

SEQ ID NO: 36 - protein, 13 aa, unidentified
ITVVDALHEIPVK

SEQ ID NO: 37 - protein, 16 aa, unidentified
TELLPGDRDNLAIQTR

SEQ ID NO: 38 - protein, 9 aa, unidentified
ANVFVQLPR

SEQ ID NO: 39 - protein, 14 aa, unidentified
LAELPADALGPLQR

SEQ ID NO: 40 - protein, 22 aa, unidentified
SFEGLGQLEVLTLDHNQLQEVK

SEQ ID NO: 41 - protein, 11 aa, unidentified
TFTPQPPGLER

SEQ ID NO: 42 - protein, 16 aa, unidentified
VAGLLEDTFPGLLGLR

SEQ ID NO: 43 - protein, 18 aa, unidentified
VTGLDFIPGLHPILTLSK

SEQ ID NO: 44 - protein, 15 aa, unidentified
DFDEEVAIVEELGRR

SEQ ID NO: 45 - protein, 20 aa, unidentified
HAEGTFTSDVSSYLEGQAAK

SEQ ID NO: 46 - protein, 14 aa, unidentified
RAPQTGIVDECCFR

(19) United States
(12) Patent Application Publication
(10) Pub. No.: US 2025/0283891 A1
(43) Pub. Date: Sep. 11, 2025

(54) METHODS FOR DETECTING METABOLIC MARKERS

(71) Applicants: Cedars-Sinai Medical Center, Los Angeles, CA (US); Seer, Inc., Redwood City, CA (US)

(72) Inventors: Simion Kreimer, Los Angeles, CA (US); Jennifer Van Eyk, Los Angeles, CA (US); Kimia Sobhani, Los Angeles, CA (US); Khatereh Motamedchaboki, Cupertino, CA (US); Aaron Gajadhar, Redwood City, CA (US); Ryan Benz, Huntington Beach, CA (US); Shao-Yung Chen, Mountain View, CA (US)

(21) Appl. No.: 19/221,165

(22) Filed: May 28, 2025

Related U.S. Application Data
(63) Continuation of application No. PCT/US2023/081697, filed on Nov. 29, 2023.
(60) Provisional application No. 63/385,589, filed on Nov. 30, 2022.

(51) Int. Cl. G01N 33/68; C12Q 1/37; G01N 33/543; G01N 33/66

(52) U.S. Cl. CPC G01N 33/6848; C12Q 1/37; G01N 33/54326; G01N 33/66; G01N 2333/4745; G01N 2333/605; G01N 2333/65; G01N 2333/976; G01N 2400/00

(57) ABSTRACT

Disclosed herein are methods for analyzing a biological sample. The methods of the present disclosure may comprise incubating the biological sample to form a protein corona and assaying protein in the corona to identify one or more analyte proteins using a targeted method.

### CROSS-REFERENCE

This application is a continuation of International Application No. PCT/US2023/081697, filed Nov. 29, 2023, which claims the benefit of U.S. Provisional Application No. 63/385,589, filed Nov. 30, 2022, each of which is incorporated herein by reference in its entirety.

### STATEMENT AS TO FEDERALLY SPONSORED RESEARCH

The present disclosure was made with government support under U01 DK124019-01 awarded by the National Institutes of Health. The government has certain rights in the disclosure.

### INCORPORATION BY REFERENCE OF SEQUENCE LISTING

The present application is being filed along with a Sequence Listing in electronic format. The Sequence Listing is provided as a file entitled 53344-771_301_SL.xml, created May 27, 2025, which is 40,745 bytes in size. The information in the electronic format of the Sequence Listing is incorporated by reference in its entirety.

### BACKGROUND

Metabolism is regulated through the exquisite interplay of numerous biomolecular and biochemical actors, such as hormones, which maintain homeostatic balance in an organism. Recent links between cell signaling, metabolism, and pathogenesis of disease have been elucidated. Nevertheless, rapid, multiplexed (e.g., multiple species simultaneously) quantitative analysis of key metabolic hormones and regulatory proteins remains evasive using documented methods.

### SUMMARY

An aspect of the present disclosure provides a method of analyzing a biological sample, comprising: incubating the biological sample with a surface to form a protein corona; and assaying proteins in the protein corona using a targeted method to determine a presence or absence of one or more analyte proteins, wherein the one or more analyte proteins is selected from insulin-like growth factor I (IGF-1), insulin-like growth factor II (IGF-2), IGF binding protein 1 (IGFBP1), IGF binding protein 2 (IGFBP2), IGF binding protein 3 (IGFBP3), IGF binding protein 4 (IGFBP4), IGF binding protein 5 (IGFBP5), IGF binding protein 6 (IGFBP6), IGF binding protein 7 (IGFBP7), Insulin-like growth factor binding protein acid labile subunit (IGFALS), glucagon-like peptide 1 (GLP-1), glucagon-like peptide 2 (GLP-2), leptin, and any combination thereof.

In some embodiments, the surface comprises a surface functionalization. In some embodiments, the surface functionalization comprises an amine functionalization. In some embodiments, the surface functionalization comprises a dimethyl amino functionalization. In some embodiments, the surface functionalization comprises a sugar. In some embodiments, the sugar comprises a sugar phosphate. In some embodiments, the sugar comprises glucose. In some embodiments, the sugar comprises glucose 6-phosphate. In some embodiments, the sugar is a monosaccharide. In some embodiments, the surface is a surface of a particle. In some embodiments, the particle is a nanoparticle. In some embodiments, the particle is a microparticle. In some embodiments, the particle has an average diameter of less than 500 nm. In some embodiments, the particle comprises a magnetic material. In some embodiments, the particle is a superparamagnetic iron oxide particle. In some embodiments, the particle comprises an iron oxide material. In some embodiments, the particle comprises an iron oxide core. In some embodiments, the particle comprises an iron oxide particle embedded in a polystyrene core. In some embodiments, the particle comprises a polymer coating. In some embodiments, the polymer coating comprises the surface functionalization. In some embodiments, the particle comprises a polymethacrylamide or polyacrylamide coating comprising an amine functional group. In some embodiments, the particle comprises a poly(N-(3-(dimethylamino) propyl) methacrylamide) (PDMAPMA) coating. In some embodiments, the particle comprises a positive zeta potential. In some embodiments, the zeta potential has a magnitude greater than about 10 millivolts (mV) or great than about 20 mV. In some embodiments, the zeta potential has a magnitude less than about 40 mV or about 40 mV. In some embodiments, the zeta potential has a magnitude from about 20 mV to about 40 mV. In some embodiments, the particle comprises a negative zeta potential. In some embodiments, the zeta potential has a magnitude greater than about 10 millivolts or about 20 (mV). In some embodiments, the zeta potential has a magnitude less than about 50 mV or about 40 mV. In some embodiments, the surface is comprised of a plurality of surfaces, and wherein the incubating comprises contacting the biological sample with the plurality of surfaces. In some embodiments, the plurality of surfaces have the same surface functionalization. In some embodiments, at least 90% by area of the plurality of surfaces have the same surface functionalization. In some embodiments, at least 97% by area of the plurality of surfaces have the same surface functionalization. In some embodiments, the plurality of surfaces is a plurality of surfaces of particles. In some embodiments, the particles are characterized by a polydispersity index (PDI) less than about 0.1. In some embodiments, a second surface of the plurality of surfaces comprises a second surface functionalization. In some embodiments, each surface of the plurality of surfaces comprises the surface functionalization. In some embodiments, the assaying comprises quantifying an amount of the one or more analyte proteins. In some embodiments, the assaying comprises targeted mass spectrometry. In some embodiments, the targeted mass spectrometry comprises tandem mass spectrometry (MS/MS). In some embodiments, the assaying comprises liquid chromatography-tandem mass spectrometry (LC-MS/MS). In some embodiments, the assaying comprises multiple reaction monitoring (MRM), parallel reaction monitoring (PRM), or selected reaction monitoring (SRM). In some embodiments, the analyte proteins are detected using a single mass spectrometry injection. In some embodiments, the assaying comprises affinity-based detection. In some embodiments, the assaying comprises peptide or aptamer binding. In some embodiments, the method further comprises, prior to the assaying the analyte proteins, separating at least a portion of the analyte proteins from the surface. In some embodiments, the method further comprises, digesting at least a portion of the biomolecules adsorbed to the surface. In some embodiments, the digesting comprises digesting the at least a portion of the biomolecules with a protease (e.g., trypsin). In some embodiments, the assaying comprises detecting or quantifying one or more peptides derived from the one or more analyte proteins. In some embodiments, the one or more peptides are selected from the peptides in Table 1 or Table 2. In some embodiments, the biological sample is blood, plasma, or serum. In some embodiments, the assaying comprises detecting or quantifying IGF1 and IGF2. In some embodiments, the assaying comprises detecting or quantifying GLP-1 and leptin. In some embodiments, the assaying comprises detecting or quantifying at least one of IGFBP1, IGFBP2, IGFBP3, IGFBP4, IGFBP5, IGFBP6, or IGFBP7, or a derivative thereof. In some embodiments, the assaying comprises detecting or quantifying at least two IGFBP1, IGFBP2, IGFBP3, IGFBP4, IGFBP5, IGFBP6, or IGFBP7, or a derivative thereof. In some embodiments, the assaying comprises detecting or quantifying at least three IGFBP1, IGFBP2, IGFBP3, IGFBP4, IGFBP5, IGFBP6, or IGFBP7, or a derivative thereof. In some embodiments, the assaying comprises detecting or quantifying at least four of IGFBP1, IGFBP2, IGFBP3, IGFBP4, IGFBP5, IGFBP6, or IGFBP7, or a derivative thereof. In some embodiments, the assaying comprises detecting or quantifying all of IGFBP1, IGFBP2, IGFBP3, IGFBP4, IGFBP5, IGFBP6, or IGFBP7, or a derivative thereof. In some embodiments, the assaying comprises detecting or quantifying leptin or a derivative thereof. In some embodiments, the assaying comprises detecting or quantifying GLP-1 and leptin, or any derivative(s) thereof, among the analyte proteins. In some embodiments, the method further comprises, diagnosing, monitoring, or screening a subject for a disease or disorder based on the presence or absence of the one or more analyte proteins. In some embodiments, the diagnosing, monitoring, or screening comprises identifying the presence or absence of at least a subset of the one or more analyte proteins.

An aspect of the present disclosure provides use of a sugar-functionalized particle for targeted detection or quantification of one or more analyte proteins selected from IGF binding protein 1 (IGFBP1), IGF binding protein 2 (IGFBP2), IGF binding protein 3 (IGFBP3), IGF binding protein 4 (IGFBP4), IGF binding protein 5 (IGFBP5), IGF binding protein 6 (IGFBP6), IGF binding protein 7 (IGFBP7), and Insulin-like growth factor binding protein acid labile subunit (IGFALS) in a biological sample.

In some embodiments, the sugar-functionalized particle comprises a phosphorylated sugar functionalization. In some embodiments, the sugar-functionalized particle comprises a phosphorylated monosaccharide functionalization. In some embodiments, the sugar-functionalized particle comprises a glucose 6-phosphate (G6P) functionalization.

An aspect of the present disclosure provides use of a sugar-functionalized particle for quantifying an amount of IGF-1 and IGF-2 in a biological sample or fraction thereof.

In some embodiments, the sugar-functionalized particle comprises a phosphorylated sugar functionalization. In some embodiments, the sugar-functionalized particle comprises a phosphorylated monosaccharide functionalization. In some embodiments, the sugar-functionalized particle comprises a glucose 6-phosphate (G6P) functionalization.

An aspect of the present disclosure provides use of a polymethacrylamide or polyacrylamide coated particle for targeted detection or quantification of glucagon-like peptide 1 (GLP-1) and/or leptin, wherein the polymethacrylamide or polyacrylamide comprises an amine functional group.

An aspect of the present disclosure provides a method of analyzing a plasma or serum sample, comprising: incubating the plasma or serum sample with a phosphorylated-sugar functionalized particle to form a protein corona; and assaying proteins in the protein corona using targeted mass spectrometry to quantify insulin-like growth factor I (IGF-1) and insulin-like growth factor II (IGF-2).

In some embodiments, the assaying further comprises quantifying one or more analyte proteins selected from the group consisting of IGF binding protein 1 (IGFBP1), IGF binding protein 2 (IGFBP2), IGF binding protein 3 (IGFBP3), IGF binding protein 4 (IGFBP4), IGF binding protein 5 (IGFBP5), IGF binding protein 6 (IGFBP6), IGF binding protein 7 (IGFBP7), and Insulin-like growth factor binding protein acid labile subunit (IGFALS). In some embodiments, at least two, at least three, at least four, at least five, at least six, at least seven, or all of the analyte proteins are quantified. In some embodiments, the particle is functionalized with a phosphorylated monosaccharide.

An aspect of the present disclosure provides a method of analyzing a plasma or serum sample, comprising: incubating said plasma or serum sample with a polymethacrylamide or polyacrylamide coated particle to form a protein corona, wherein said polymethacrylamide or polyacrylamide comprises an amine functional group; and assaying proteins in the protein corona using targeted mass spectrometry to quantify glucagon-like peptide 1 (GLP-1) or leptin.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A depicts graphs showing total proteins and peptide precursors identified by methods of the present disclosure.

FIG. 1B depicts a Venn diagram showing counts of unique proteins identified in the biomolecule coronas formed on several different surfaces of the present disclosure.

FIG. 2A depicts a graph showing quantification of analyte proteins as described herein across several replicates of a biomolecule corona analysis.

FIG. 2B depicts graphs showing quantification of insulin-like growth factor 1 (IGF-1) (left) and insulin-like growth factor 2 (IGF-2) (right) across several human plasma replicates of a biomolecule corona analysis as described herein.

FIG. 3A depicts a graph showing coefficients of variation (CVs) for several representative peptide derivatives of IGF-1 (a) and IGF-2 (b) detected across replicates of the biomolecule corona analysis. Figure discloses SEQ ID NOS 3-6, and 6, respectively, in order of appearance.

FIG. 3B depicts a graph showing quantification of insulin-like growth factor binding partners 1 through 7 (IGFBP1-7) and IGF binding protein acid labile subunit (IGFALS) across several replicates of a biomolecule corona analysis as described herein (left panel). FIG. 3B also depicts a table showing CVs for the quantified proteins across each replicate of the biomolecule corona analysis (right panel).

FIG. 4A depicts graphs showing coefficients of variation (CVs) for several representative peptide derivatives of glucagon-like peptide 1 (GLP-1) detected across replicates of a biomolecule corona analysis. Figure discloses SEQ ID NOS 44-45, and 43, respectively, in order of appearance.

FIG. 4B depicts graphs showing coefficients of variation (CVs) for several representative peptide derivatives of leptin detected across replicates of a biomolecule corona analysis. Figure discloses SEQ ID NOS 44-45, and 43, respectively, in order of appearance.

### DETAILED DESCRIPTION

Disclosed herein are methods of identifying and quantifying metabolic biomarkers through plasma protein profiling. The methods disclosed herein can achieve superior metabolic biomarker identification and/or quantification using a novel proteomics profiling platform of functionalized particles in combination with mass spectrometry (MS). Particles (e.g., nanoparticles) specifically and reproducibly interrogate (e.g., isolate) subsets of protein from biofluids and have high efficiency and effectiveness for proteomics profiling, such as by quantification and/or identification by MS.

Insulin-like growth factors are involved in growth, obesity, and other metabolic disorders. Insulin-like growth factor 1 (IGF-1) regulates the effects of growth hormone (GH) promoting adult normal growth of bones and tissues. GH levels in the blood fluctuate depending on diet and activity, while IGF-1 levels generally remain stable in an individual. Insulin-like growth factor 2 (IGF-2) is the major fetal isoform of IGF, but it is present in adults and its abundance is altered in metabolic and other conditions (e.g., obesity, diabetes, polycystic ovary syndrome). Circulating IGF-1 and IGF-2 are bound to other proteins, such as insulin-like growth factor binding protein acid label subunit (IGFALS) and insulin-like growth factor binding proteins 1-7 (IGFBP1, IGFBP2, IGFBP3, IGFBP4, IGFBP5, IGFBP6, IGFBP7), which influence their biological function, and can be altered (e.g., in abundance) in disease states.

Despite the clinical relevance of IGF isoforms 1 and 2 and their binding partners, accurate identification, and quantification of these biomarkers from biological samples with affinity-based (e.g., antibody) assays are plagued by difficulties.

Glucagon-like peptide 1 (GLP-1) is a naturally-occurring incretin hormone released into the circulation by the L cells of the gut in response to ingested nutrients. By binding to its cognate receptor (GLP-1R) GLP-1 is able to promote insulin secretion while suppressing glucagon secretion. GLP-1 can be difficult to quantify in biological samples as it is rapidly degraded by naturally occurring proteases.

Leptin is a hormone released by adipose tissue which is involved in regulating satiety. Several documented conditions are correlated with leptin dysregulation or dysfunction, such as leptin resistance, hyperinsulinemia, fatty liver disease, dyslipidemia, and hypogonadotropic hypogonadism. Despite this fact, leptin is not routinely tested due to technical challenges.

The biomarkers (e.g., circulating biomarkers) disclosed herein may be identified and quantified using particle panels having one or more different particle types, which are incubated with samples to form biomolecule coronas on the surface of the particles and assayed for proteins in the biomolecule coronas. Particle panels can have multiple distinct particle types, which enrich proteins from a sample onto distinct biomolecule coronas formed on the surface of the distinct particle types. The combinations of particle types selected for inclusion in a particle panel of the present disclosure can be varied in their physicochemical properties (e.g., size, surface charge, core material, shell material, surface chemistry, porosity, morphology, and other properties). However, particle types may also share one or more physicochemical properties. Particles can be engineered to possess certain physicochemical properties, such as size and surface functionalization, which reproducibly interact with (e.g., enrich) distinct protein populations to generated characteristic biomolecule coronas. The term “biomolecule corona,” as used herein generally refers to the formation of a layer of biomolecules (e.g., proteins) on the surface of a particle after the particle has been contacted with a sample (e.g., plasma). Further, proteins that are not detectable in plasma using conventional mass spectrometry can be reliably identified in and quantified from these coronas. Particle types included in the particle panels disclosed herein may be particularly suited for the identification and quantification of metabolic biomarkers which are difficult to detect by other (e.g., affinity-based, such as antibody binding) assays. Further, methods of the present disclosure may allow rapid, specific quantification of metabolic biomarkers. Additionally, methods of the present disclosure may allow quantification of pluralities of biomarkers simultaneously which may further provide more precise mechanistic insight into diseases associated with these biomarkers.

### Definitions

Unless defined otherwise, all technical and scientific terms used herein have the same meaning as is commonly understood by one of skill in the art to which this disclosure belongs. All patents and publications referred to herein are incorporated by reference.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, a “feature” identified by mass spectrometry includes a signal at a specific combination of retention time and m/z (mass-to-charge ratio), where each feature has an associated intensity. Some features are further fragmented in a second mass spectrometry analysis (MS2) for identification.

As used herein, the term “biomolecule corona” generally refers to the plurality of different biomolecules that bind to a sensor element. The term “biomolecule corona” generally refers to the proteins, lipids and other plasma components that bind to particles (e.g., nanoparticles) when they come into contact with biological samples or biological system. For use herein, the term “biomolecule corona” also encompasses both the soft and hard protein corona as referred to in Milani et al. “Reversible versus Irreversible Binding of Transferring to Polystyrene Nanoparticles: Soft and Hard Corona” ACS NANO, 2012, 6 (3), pp. 2532-2541; Mirshafiee et al. “Impact of protein pre-coating on the protein corona composition and nanoparticle cellular uptake” Biomaterials vol. 75, January 2016 pp. 295-304, Mahmoudi et al. “Emerging understanding of the protein corona at the nano-bio interfaces” Nanotoday 11 (6) December 2016, pp. 817-832, and Mahmoudi et al. “Protein-Nanoparticle Interactions: Opportunities and Challenges” Chem. Rev., 2011, 111 (9), pp. 5610-5637, the contents of which are incorporated by reference in their entireties. As described therein, an adsorption curve may show the build-up of a strongly bound monolayer up to the point of monolayer saturation (at a geometrically defined protein-to-NP ratio), beyond which a secondary, weakly bound layer is formed. While the first layer is irreversibly bound (hard corona), the secondary layer (soft corona) may exhibit dynamic exchange. Proteins that adsorb with high affinity may form the “hard” corona, comprising tightly bound proteins that do not readily desorb, and proteins that adsorb with low affinity may form the “soft” corona, comprising loosely bound proteins. Soft and hard corona can also be characterized based on their exchange times. Hard corona may show much larger exchange times in the order of several hours. See, e.g., M. Rahman et al. Protein-Nanoparticle Interactions, Spring Series in Biophysics 15, 2013, incorporated by reference in its entirety.

The term “biomolecule” generally refers to biological components that may be involved in corona formation, including, but not limited to, for example, proteins, polypeptides, polysaccharides, a sugar, a lipid, a lipoprotein, a metabolite, an oligonucleotide, metabolome or combination thereof. It is contemplated that the biomolecule coronas of distinct particles may contain some of the same biomolecules, may contain distinct biomolecules with regard to the other sensor elements, and/or may differ in level or quantity, type or conformation of the biomolecule that binds to each sensor element. In one embodiment, the biomolecule is selected from the group of proteins, nucleic acids, lipids, and metabolomes.

The term “biomolecule corona signature” generally refers to the composition, signature or pattern of different biomolecules that are bound to each type of particle or separate sensor element. The signature may not only refer to the different biomolecules but also the differences in the amount, level or quantity of the biomolecule bound to the sensor element, or differences in the conformational state of the biomolecule that is bound to the particle or sensor element. It is contemplated that the biomolecule corona signatures of each distinct type of sensor elements may contain some of the same biomolecules, may contain distinct biomolecules with regard to the other sensor elements, and/or may differ in level or quantity, type, or conformation of various biomolecules. The biomolecule corona signature may depend on not only the physicochemical properties of the sensor element (e.g., particle), but also the nature of the sample and the duration of exposure to the biological sample.

“Biomolecule” as used in “biomolecule corona” can refer to any molecule or biological component that can be produced by, or is present in, a biological organism. Non-limiting examples of biomolecules include proteins (protein corona), polypeptides, oligopeptides, polyketides, polysaccharides, a sugar, a lipid, a lipoprotein, a metabolite, an oligonucleotide, a nucleic acid (DNA, RNA, micro RNA, plasmid, single stranded nucleic acid, double stranded nucleic acid), metabolome, as well as small molecules such as primary metabolites, secondary metabolites, and other natural products, or any combination thereof. In some embodiments, the biomolecule is selected from the group of proteins, nucleic acids, lipids, and metabolomes.

As used herein, the term “sensor element” refers to elements that are able to bind to a plurality of biomolecules when in contact with a sample and encompasses the term “nanoscale sensor element”. A sensor element may be a particle, such as a nanoparticle, or microparticle. A sensor element may be a surface or a portion of a surface. A sensor element may comprise a particle or plurality of particles. A sensor element may comprise a plurality of surfaces capable of adsorbing or binding biomolecules. A sensor element may comprise a porous material, such as a material into which biomolecules can intercalate.

As used herein, a “sensor array” may comprise a plurality of sensor elements wherein the plurality of sensor elements (e.g., particles) comprises multiple types of sensor elements. The sensor elements may be different types that differ from each other in at least one physicochemical property. A sensor array may be a substrate with differentially modified surface regions. A sensor array may be a substrate with a plurality of partitions containing a plurality of sensor elements (e.g., particles). For example, a sensor array may comprise a multi-well plate with a plurality of particles distributed between the plurality of wells. A sensor array may be a substrate comprising a plurality of partitions, wherein the plurality of partitions comprises a plurality of particles. In some cases, each sensor element or particle is able to bind a plurality of biomolecules in a sample to produce a biomolecule corona signature. In some embodiments, each sensor element (e.g., particle type) has a distinct biomolecule corona signature.

### Methods for Assaying Biological Samples

The present disclosure provides methods for assaying biological samples. In some cases, the methods comprise an operation of incubating the biological sample with a surface to form a protein corona. The surface may comprise a surface functionalization as described herein. As described in more detail herein below, the particular surface functionalization of the surface may preferentially bind certain proteins in the protein corona which comprise one or more metabolic analyte proteins (e.g., biomarkers). In some cases, the methods further comprise an operation of assaying proteins in the protein corona with a targeted (e.g., mass spectrometry) assay to determine the presence or absence of and/or quantify one or more analyte proteins, wherein the analyte proteins are selected from insulin-like growth factor I (IGF-1), insulin-like growth factor II (IGF-2), IGF binding protein 1 (IGFBP1), IGF binding protein 2 (IGFBP2), IGF binding protein 3 (IGFBP3), IGF binding protein 4 (IGFBP4), IGF binding protein 5 (IGFBP5), IGF binding protein 6 (IGFBP6), IGF binding protein 7 (IGFBP7), Insulin-like growth factor binding protein acid labile subunit (IGFALS), glucagon-like peptide 1 (GLP-1), glucagon-like peptide 2 (GLP-2), leptin, and any combination thereof. The protein can be intact or (e.g., post translationally) modified, including proteolyzed, glycosylation or phosphorylation, for example. Optionally, methods may comprise an operation of diagnosing, monitoring, or screening a subject for a disease or disorder based on said presence or absence of said one or more analyte proteins.

The present disclosure also provides functionalized surfaces (e.g., particles) for use in assaying (e.g., identifying or quantifying) analyte proteins as disclosed herein. Without wishing to be bound by a particular theory, the functionalization of the particles may comprise moieties or physiochemical properties which preferentially interact with one or more analyte proteins as disclosed herein. For example, in some cases, the functionalized particles comprise a sugar (e.g., monosaccharide, such as glucose) functionalization. The analyte proteins as disclosed herein may comprise one or more sequences or structural motifs which preferentially interact with sugars. For example, an analyte protein as disclosed herein may comprise a sugar binding motif which comprises one or more aromatic amino acids (e.g., tryptophan, tyrosine, histidine, phenylalanine) which have an aromatic π electron system (π system) which may interact strongly with the hydrogen atoms present in C—H or O—H bonds of sugar molecules. In another example, an analyte protein as disclosed herein may comprise a sugar binding motif which comprises one or more polar amino acids (e.g., aspartic acid, glutamic acid, asparagine, glutamine, arginine, histidine, lysine, serine, threonine) which interact strongly with the highly-polarized O—H and C—H bonds in sugar molecules. In some cases, an analyte protein interacts with a surface comprising a sugar functionalization through other driving forces or physicochemical properties as discussed elsewhere herein.

In some cases, the analyte proteins as disclosed herein may comprises one or more sequence or structural motifs which preferentially interact with phosphorylated sugars. For example, in some cases, the functionalized particles comprise a phosphorylated sugar (e.g., glucose 6-phosphate (G6P)) functionalization. The analyte proteins as disclosed herein may comprise one or more sequence or structural motifs which preferentially interact with phosphorylated sugars. For example, an analyte protein as disclosed herein may comprise a phosphorylated sugar binding motif which comprises one or more aromatic amino acids (e.g., tryptophan, tyrosine, histidine, phenylalanine) which have an aromatic π electron system (I system) which may interact strongly with the hydrogen atoms present in C—H or O—H bonds of sugar molecules. In another example, an analyte protein as disclosed herein may comprise a phosphorylated sugar binding motif which comprises one or more polar amino acids (e.g., aspartic acid, glutamic acid, asparagine, glutamine, arginine, histidine, lysine, serine, threonine) which interact strongly with the highly-polarized O—H and C—H bonds in phosphorylated sugar molecules. In yet another example, an analyte protein as disclosed herein may comprise a positively charged amino acid (e.g., arginine, lysine, histidine) which preferentially interacts with the negative charge at physiological pH of a phosphate group of a phosphorylated sugar functionalization. In some cases, an analyte protein interacts with a surface comprising a phosphorylate sugar functionalization through other driving forces or physicochemical properties as discussed elsewhere herein.

In some cases, the analyte proteins as disclosed herein may comprise one or more sequence or structural motifs which preferentially interact with amine groups (e.g., comprised on a surface, such as in a polymer). For example, in some cases, the functionalized particles comprise a dimethyl amino functionalization. In some cases, the diamine functionalization comprises a polyacrylamide or polymethylacrylamide coating comprising an amine functional group. In some cases, the polymer is a poly(N-(3-(dimethylamino) propyl) methacrylamide) (PDMAPMA) coating of a particle as disclosed herein. The analyte proteins as disclosed herein may comprise one or more sequence or structural motifs which preferentially interact with amino groups. For example, an analyte protein as disclosed herein may comprise a binding motif which comprises one or more polar amino acids (e.g., aspartic acid, glutamic acid, asparagine, glutamine, arginine, histidine, lysine, serine, threonine) which interact strongly with amino groups which are charged under certain conditions (e.g., at physiological or acidic pH). In another example, an analyte protein as disclosed herein may comprise a negatively charged amino acid and/or amino acid comprising a labile proton (e.g., aspartate, glutamate, cysteine) which preferentially interacts with the positive charge of an amino group under certain conditions. In some cases, an analyte protein interacts with a surface comprising an amine functionalization through other driving forces or physicochemical properties as discussed elsewhere herein.

In some cases, the combination of incubating the surface with the biological sample and targeted assaying (e.g., with a targeted mass spectrometry technique as discussed elsewhere herein) of the resulting protein corona may allow for identification (e.g., determining the presence or absence of) and/or quantification (e.g., determining a relative or absolute amount of) of one or more metabolic analyte proteins as disclosed herein. In an example, a surface may comprise a sugar functionalization which is able to adsorb one or more insulin-like growth factor proteins, such as IGF-1 and IGF-2 and/or their binding partners, insulin-like growth factor binding proteins 1-7 (IGFBP1-7) and insulin-like growth factor binding protein acid labile subunit. In another example, a surface may comprise an amine functionalization which is able to adsorb one or more hormones involved in sugar metabolism or satiety, such as glucagon-like peptide 1 (GLP-1) or lectin. Combined with a targeted assay (e.g., targeted mass spectrometry method, such as those disclosed herein), methods of the present disclosure may be able to identify and/or quantify panels of such metabolic analyte proteins. The methods of the present disclosure may be able to assay the metabolic analyte proteins rapidly and/or simultaneously, as compared to other methods for assaying such analyte proteins.

### Analyte Proteins

Analyte proteins (e.g., biomarkers) which are detected and/or quantified by the methods disclosed herein may comprise at least one of the following: insulin-like growth factor I (IGF-1), insulin-like growth factor II (IGF-2), IGF binding protein 1 (IGFBP1), IGF binding protein 2 (IGFBP2), IGF binding protein 3 (IGFBP3), IGF binding protein 4 (IGFBP4), IGF binding protein 5 (IGFBP5), IGF binding protein 6 (IGFBP6), IGF binding protein 7 (IGFBP7), insulin-like growth factor binding protein acid labile subunit (IGFALS), glucagon-like peptide 1 (GLP-1), glucagon-like peptide 2 (GLP-2), and leptin. In some embodiments, the analyte proteins comprise insulin-like growth factor I (IGF-1). In some cases, the analyte proteins comprise insulin-like growth factor II (IGF-2). In some cases, the analyte proteins comprise IGF binding protein 1 (IGFBP1). In some cases, the analyte proteins comprise IGF binding protein 2 (IGFBP2). In some cases, the analyte proteins comprise IGF binding protein 3 (IGFBP3). In some cases, the analyte proteins comprise IGF binding protein 4 (IGFBP4). In some cases, the analyte proteins comprise IGF binding protein 5 (IGFBP5). In some cases, the analyte proteins comprise IGF binding protein 6 (IGFBP6). In some cases, the analyte proteins comprise IGF binding protein 7 (IGFBP7). In some cases, the analyte proteins comprise insulin-like growth factor binding protein acid labile subunit (IGFALS). In some cases, the analyte proteins comprise glucagon-like peptide 1 (GLP-1). In some cases, the analyte proteins comprise glucagon-like peptide 2 (GLP-2). In some cases, the analyte proteins comprise leptin.

In some cases, detecting or quantifying an analyte protein comprises detecting or quantifying a derivative of the analyte proteins. In some cases, detecting or quantifying the analyte protein comprises detecting or quantifying a peptide fragment derived from digestion of the analyte protein. In some cases, the digestion may comprise chemical digestion, such as by cyanogen bromide or 2-Nitro-5-thiocyanatobenzoic acid (NTCB). In some cases, the digestion may comprise enzymatic digestion, such as by a protease (e.g., trypsin or pepsin). In some cases, digestion may occur in the biological sample or in the subject form which the biological sample is derived in response to physiological or pathological (e.g., disease) state or status. In some cases, the digestion is performed as part of one or more sample processing operations. In some cases, the digestion may cleave peptides at a specific position (e.g., at methionines) or sequence (e.g., glutamate-histidine-glutamate). In some cases, the digestion may enable similar proteins to be distinguished. For example, an assay may resolve 8 distinct proteins as a single protein group with a first digestion method, and as 8 separate proteins with distinct signals with a second digestion method. In some cases, the digestion may generate an average peptide fragment length of 8 to 15 amino acids. In some cases, the digestion may generate an average peptide fragment length of 12 to 18 amino acids. In some cases, the digestion may generate an average peptide fragment length of 15 to 25 amino acids. In some cases, the digestion may generate an average peptide fragment length of 20 to 30 amino acids. In some cases, the digestion may generate an average peptide fragment length of 30 to 50 amino acids. In some cases, the digestion may generate a peptide comprising a sequence of DFPFFVAIVFFLGRR (SEQ ID NO: 1). In some cases, the digestion may generate a peptide comprising a sequence of HAFGTFTSDVSSYLEGQAAK (SEQ ID NO: 2). In some cases, the digestion may generate a peptide comprising a sequence of APQTGIVDECCFR (SEQ ID NO: 3). In some cases, the digestion may generate a peptide comprising a sequence of LEMYCAPLK (SEQ ID NO: 4). In some cases, the digestion may generate a peptide comprising a sequence of GIVEECCFR (SEQ ID NO: 5). In some cases, the digestion may generate a peptide comprising a sequence of SCDLALLETYCATPAK (SEQ ID NO: 6).

In some cases, cysteine residues in a protein or peptide may be chemically derivatized (e.g., carbamidomethylated). Chemical derivatization of cysteine residues may prevent side reactions of the nucleophilic sulfhydryl sides chains, such as the reformation of disulfide bonds within or between proteins or peptides. In some cases, the cysteine is alkylated (e.g., with an alkylating agent). In some cases, the alkylating agent is iodoacetate, iodoacetamide, chloroacetamide, or acrylamide. some cases, cysteine residues are not chemically derivatized.

In some cases, the digestion may generate a peptide comprising one or more of the sequences listed in Table 1 or Table 2. In some cases, detecting and/or quantifying analyte proteins comprises detecting and/or quantifying one or more of the peptides in Table 1 or Table 2.

TABLE 1
nsulin-like growth factor binding protein peptides

| Gene | Protein Name | Peptide Sequence | Charge |
|---|---|---|---|
| IGFBP1 | Insulin-like growth factor-binding protein 1 | SAGCGCCPMCALPLGAACGVATAR | 3 |
| | | (SEQ ID NO: 7) | |
| IGFBP2 | Insulin-like growth factor-binding protein 2 | CPPCTPER (SEQ ID NO: 8) | 2 |
| IGFBP2 | Insulin-like growth factor-binding protein 2 | EPGCGCCSVCAR (SEQ ID NO: 9) | 2 |
| IGFBP2 | Insulin-like growth factor-binding protein 2 | GDPECHLFYNEQQEAR (SEQ ID NO: | 3 |
| | | 10) | |
| IGFBP2 | Insulin-like growth factor-binding protein 2 | GPLEHLYSLHIPNCDK (SEQ ID NO: | 3 |
| | | 11) | |
| IGFBP2 | Insulin-like growth factor-binding protein 2 | GPLEHLYSLHIPNCDK (SEQ ID NO: | 4 |
| | | 11) | |
| IGFBP2 | Insulin-like growth factor-binding protein 2 | LAACGPPPVAPPAAVAAVAGGAR | 3 |
| | | (SEQ ID NO: 12) | |
| IGFBP2 | Insulin-like growth factor-binding protein 2 | LEGEACGVYTPR (SEQ ID NO: 13) | 2 |
| IGFBP2 | Insulin-like growth factor-binding protein 2 | LIQGAPTIR (SEQ ID NO: 14) | 2 |
| IGFBP2 | Insulin-like growth factor-binding protein 2 | TPCQQELDQVLER (SEQ ID NO: 15) | 2 |
| IGFBP2 | Insulin-like growth factor-binding protein 2 | TPCQQELDQVLER (SEQ ID NO: 15) | 3 |
| IGFBP3 | Insulin-like growth factor-binding protein 3 | ALAQCAPPPAVCAELVR (SEQ ID | 3 |
| | | NO: 16) | |
| IGFBP3 | Insulin-like growth factor-binding protein 3 | CQPSPDEARPLQALLDGR (SEQ ID | 3 |
| | | NO: 17) | |
| IGFBP3 | Insulin-like growth factor-binding protein 3 | ETEYGPCR (SEQ ID NO: 18) | 2 |
| IGFBP3 | Insulin-like growth factor-binding protein 3 | FLNVLSPR (SEQ ID NO: 19) | 2 |
| IGFBP3 | Insulin-like growth factor-binding protein 3 | GLCVNASAVSR (SEQ ID NO: 20) | 2 |
| IGFBP3 | Insulin-like growth factor-binding protein 3 | GVHIPNCDK (SEQ ID NO: 21) | 2 |
| IGFBP3 | Insulin-like growth factor-binding protein 3 | SAGSVESPSVSSTHR (SEQ ID NO: | 3 |
| | | 22) | |
| IGFBP3 | Insulin-like growth factor-binding protein 3 | YGQPLPGYTTK (SEQ ID NO: 23) | 2 |
| IGFBP4 | Insulin-like growth factor-binding protein 4 | EDARPVPQGSCQSELHR (SEQ ID | 3 |
| | | NO: 24) | |
| IGFBP4 | Insulin-like growth factor-binding protein 4 | EDARPVPQGSCQSELHR (SEQ ID | 4 |
| | | NO: 24) | |
| IGFBP4 | Insulin-like growth factor-binding protein 4 | GELDCHQLADSFRE (SEQ ID NO: 25) | 3 |
| IGFBP4 | Insulin-like growth factor-binding protein 4 | LPGGLEPK (SEQ ID NO: 26) | 2 |
| IGFBP4 | Insulin-like growth factor-binding protein 4 | THEDLYIIPIPNCDR (SEQ ID NO: 27) | 3 |
| IGFBP5 | Insulin-like growth factor-binding protein 5 | AVYLPNCDR (SEQ ID NO: 28) | 2 |
| IGFBP5 | Insulin-like growth factor-binding protein 5 | AVYLPNCDRK (SEQ ID NO: 29) | 3 |
| IGFBP5 | Insulin-like growth factor-binding protein 5 | FVGGAENTAHPR (SEQ ID NO: 30) | 3 |
| IGFBP6 | Insulin-like growth factor-binding protein 6 | APAVAEENPK (SEQ ID NO: 31) | 2 |
| IGFBP6 | Insulin-like growth factor-binding protein 6 | EGQECGVYTPNCAPGLQCHPPK | 3 |
| | | (SEQ ID NO: 32) | |
| IGFBP6 | Insulin-like growth factor-binding protein 6 | GAQTLYVPNCDHR (SEQ ID NO: 33) | 3 |
| IGFBP6 | Insulin-like growth factor-binding protein 6 | HLDSVLQQLQTEVYR (SEQ ID NO: | 3 |
| | | 34) | |
| IGFBP7 | Insulin-like growth factor-binding protein 7 | AGAAAGGPGVSGVCVCK (SEQ ID | 2 |
| | | NO: 35) | |
| IGFBP7 | Insulin-like growth factor-binding protein 7 | ITVVDALHEIPVK (SEQ ID NO: 36) | 3 |
| IGFBP7 | Insulin-like growth factor-binding protein 7 | TELLPGDRDNLAIQTR (SEQ ID NO: | 3 |
| | | 37) | |
| IGFALS | IGF-BP complex acid labile subunit | ANVFVQLPR (SEQ ID NO: 38) | 2 |
| IGFALS | IGF-BP complex acid labile subunit | LAELPADALGPLQR (SEQ ID NO: 39) | 2 |
| IGFALS | IGF-BP complex acid labile subunit | LAELPADALGPLQR (SEQ ID NO: 39) | 3 |
| IGFALS | IGF-BP complex acid labile subunit | SFEGLGQLEVLTLDHNQLQEVK | 3 |
| | | (SEQ ID NO: 40) | |
| IGFALS | IGF-BP complex acid labile subunit | TFTPQPPGLER (SEQ ID NO: 41) | 2 |
| IGFALS | IGF-BP complex acid labile subunit | VAGLLEDTFPGLLGLR (SEQ ID NO: | 2 |
| | | 42) | |

TABLE 2
Analyte protein peptides

| Protein | Peptide | Charge | m/z | Retention Time (min) |
|---|---|---|---|---|
| Leptin | VTGLDFIPGLHPILTLSK (SEQ | +3 | 641.0434 | 10.4-11.0 |
| | ID NO: 43) | | | |
| Proglucagon | DFDEEVAIVEELGRR (SEQ ID | +3 | 586.9722 | 8.5-9.2 |
| | NO: 44) | | | |
| GLP-1 | HAEGTFTSDVSSYLEGQAAK | +3 | 699.9956 | 5.6-6.2 |
| | (SEQ ID NO: 45) | | | |
| IGF-1 | APQTGIVDECCFR | +2 | 776.8478 | 3.6-4.2 |
| | (SEQ ID NO: 3) | | | |
| | LEMYCAPLK (SEQ ID NO: 4) | +2 | 562.7776 | 4.2-4.8 |
| | RAPQTGIVDECCFR (SEQ ID | +3 | 570.2680 | 2.6-3.2 |
| | NO: 46) | | | |
| IGF-2 | GIVEECCFR (SEQ ID NO: 5) | +2 | 585.2575 | 2.7-3.3 |
| | SCDLALLETYCATPAK (SEQ ID | +2 | 906.9290 | 6.5-7.1 |
| | NO: 6) | | | |
| | SCDLALLETYCATPAK (SEQ ID | +3 | 604.9551 | 6.5-7.1 |
| | NO: 6) | | | |

Other peptide sequences may be used to detect the analyte proteins. For example, genomic information of the subject may be used to determine peptide sequences for targeted detection or quantification (including variants of the peptides disclosed herein). Methods of using genomic information to determine suitable peptides for detection or quantification are disclosed in International Publication No. WO 2022/046804, which is hereby incorporated by reference in its entirety. As another example, suitable peptides sequences may be determined computationally using known protein sequences and predicted enzyme fragmentation.

In some cases, assaying an analyte protein comprises assaying one or more disease-induced proteolytic fragments (e.g., peptides) of the analyte protein. In some cases, assaying an analyte protein comprising assaying one or more post-translationally modified proteoforms or proteolytic fragments (e.g., peptides) of the analyte protein. In some cases, the post-translational modification comprises N-terminal extension, glycosylation, iodination, acetylation, acylation, biotinylation, amidation, alkylation, methylation, terminal amino acid cyclization, adenylation, ADP-ribosylation, sulfonation, prenylation, hydroxylation, decarboxylation, glutamylation, glycosylation, isoprenylation, lipoylation, phosphopantetheinylation, phosphorylation, or sulfation, or any combination thereof.

In some cases, detecting and/or quantifying analyte proteins comprises detecting or quantifying IGF1 and IGF2. In some cases, detecting and/or quantifying analyte proteins comprises detecting or quantifying GLP-1 and leptin. In some cases, detecting and/or quantifying analyte proteins comprises detecting or quantifying at least one of IGFBP1, IGFBP2, IGFBP3, IGFBP4, IGFBP5, IGFBP6, or IGFBP7, or a derivative thereof. In some cases, detecting and/or quantifying analyte proteins comprises detecting or quantifying at least two IGFBP1, IGFBP2, IGFBP3, IGFBP4, IGFBP5, IGFBP6, or IGFBP7, or a derivative thereof. In some cases, detecting and/or quantifying analyte proteins comprises detecting or quantifying at least three IGFBP1, IGFBP2, IGFBP3, IGFBP4, IGFBP5, IGFBP6, or IGFBP7, or a derivative thereof. In some cases, detecting and/or quantifying analyte proteins comprises detecting or quantifying at least four of IGFBP1, IGFBP2, IGFBP3, IGFBP4, IGFBP5, IGFBP6, or IGFBP7, or a derivative thereof. In some cases, detecting and/or quantifying analyte proteins comprises detecting or quantifying all of IGFBP1, IGFBP2, IGFBP3, IGFBP4, IGFBP5, IGFBP6, or IGFBP7, or a derivative thereof. In some cases, detecting and/or quantifying analyte proteins comprises detecting or quantifying leptin or a derivative thereof. In some cases, detecting and/or quantifying analyte proteins comprises detecting or quantifying GLP-1 and leptin, or any derivative(s) thereof, among said analyte proteins.

### Biological Samples

The methods of the present disclosure can be used to generate proteomic data from protein coronas from samples. Samples consistent with the present disclosure include biological samples from a subject. The subject may be a human or a non-human animal. Biological samples may be a biofluid. For example, the biofluid may be blood (e.g., whole blood), plasma, serum, CSF, urine, tear, or saliva. Biological samples can contain a plurality of proteins or proteomic data, which may be analyzed after adsorption of proteins to the surface of the various surface (e.g., sensor element, such as particle) types in a panel (e.g., one, two, or more sensor elements with different physicochemical properties) and subsequent digestion of protein coronas. Proteomic data can comprise nucleic acids, peptides, or proteins.

A wide range of biological samples are compatible for use with automated methods according to some aspects of the present disclosure. The biological sample may comprise plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. The biological sample may comprise multiple biological samples (e.g., pooled plasma from multiple subjects, or multiple tissue samples from a single subject). The biological sample may comprise a single type of biofluid or biomaterial from a single source.

The biological sample may be diluted or pre-treated. The biological sample may undergo depletion (e.g., the biological sample comprises serum) prior to use with the methods of the present disclosure. The biological sample may also undergo physical (e.g., homogenization or sonication) or chemical treatment prior to use with the methods of the present disclosure. The biological sample may be diluted prior to use with the methods of the present disclosure. The dilution medium may comprise buffer or salts, or be purified water (e.g., distilled water). Different partitions of a biological sample may undergo different degrees of dilution. A biological sample or sample partition may undergo a 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution. In some embodiments, the biological sample is plasma or serum diluted with an aqueous solution.

### Surfaces and Surface Functionalizations

### Sensor Elements

Surfaces as disclosed herein may comprise one or more surface elements. As used herein, the term “sensor element” refers to elements that are able to bind to a plurality of biomolecules when in contact with a sample and encompasses the term “particle”. Examples of suitable sensor elements include, but are not limited to, those disclosed in U.S. Pat. Nos. 11,435,360 and 11,428,688, both of which are incorporated by reference in their entirety.

The sensor element may be an element from about 5 nanometers (nm) to about 50000 nm in at least one direction. Suitable sensor elements include, for example, but not limited to a sensor element from about 5 nm to about 50,000 nm in at least one direction, including, about 5 nm to about 40000 nm, alternatively about 5 nm to about 30000 nm, alternatively about 5 nm to about 20,000 nm, alternatively about 5 nm to about 10,000 nm, alternatively about 5 nm to about 5000 nm, alternatively about 5 nm to about 1000 nm, alternatively about 5 nm to about 500 nm, alternatively about 5 nm to 50 nm, alternatively about 10 nm to 100 nm, alternatively about 20 nm to 200 nm, alternatively about 30 nm to 300 nm, alternatively about 40 nm to 400 nm, alternatively about 50 nm to 500 nm, alternatively about 60 nm to 600 nm, alternatively about 70 nm to 700 nm, alternatively about 80 nm to 800 nm, alternatively about 90 nm to 900 nm, alternatively about 100 nm to 1000 nm, alternatively about 1000 nm to 10000 nm, alternatively about 10000 nm to 50000 nm and any combination or amount in between (e.g. 5 nm, 10 nm, 15 nm, 20 nm, 25 nm, 30 nm, 35 nm, 40 nm, 45 nm, 50 nm, 55 nm, 60 nm, 65 nm, 70 nm, 80 nm, 90 nm, 100 nm, 125 nm, 150 nm, 175 nm, 200 nm, 225 nm, 250 nm, 275 nm, 300 nm, 350 nm, 400 nm, 450 nm, 500 nm, 550 nm, 600 nm, 650 nm, 700 nm, 750 nm, 800 nm, 850 nm, 900 nm, 1000 nm, 1200 nm, 1300 nm, 1400 nm, 1500 nm, 1600 nm, 1700 nm, 1800 nm, 1900 nm, 2000 nm, 2500 nm, 3000 nm, 3500 nm, 4000 nm, 4500 nm, 5000 nm, 5500 nm, 6000 nm, 6500 nm, 7000 nm, 7500 nm, 8000 nm, 8500 nm, 9000 nm, 10000 nm, 11000 nm, 12000 nm, 13000 nm, 14000 nm, 15000 nm, 16000 nm, 17000 nm, 18000 nm, 19000 nm, 20000 nm, 25000 nm, 30000 nm, 35000 nm, 40000 nm, 45000 nm, 50000 nm and any number in between). A nanoscale sensor element refers to a sensor element that is less than 1 micron in at least one direction. Suitable examples of ranges of nanoscale sensor elements include, but are not limited to, for example, elements from about 5 nm to about 1000 nm in one direction, including, from example, about 5 nm to about 500 nm, alternatively about 5 nm to about 400 nm, alternatively about 5 nm to about 300 nm, alternatively about 5 nm to about 200 nm, alternatively about 5 nm to about 100 nm, alternatively about 5 nm to about 50 nm, alternatively about 10 nm to about 1000 nm, alternatively about 10 nm to about 750 nm, alternatively about 10 nm to about 500 nm, alternatively about 10 nm to about 250 nm, alternatively about 10 nm to about 200 nm, alternatively about 10 nm to about 100 nm, alternatively about 50 nm to about 1000 nm, alternatively about 50 nm to about 500 nm, alternatively about 50 nm to about 250 nm, alternatively about 50 nm to about 200 nm, alternatively about 50 nm to about 100 nm, and any combinations, ranges or amount in-between (e.g. 5 nm, 10 nm, 15 nm, 20 nm, 25 nm, 30 nm, 35 nm, 40 nm, 45 nm, 50 nm, 55 nm, 60 nm, 65 nm, 70 nm, 80 nm, 90 nm, 100 nm, 125 nm, 150 nm, 175 nm, 200 nm, 225 nm, 250 nm, 275 nm, 300 nm, 350 nm, 400 nm, 450 nm, 500 nm, 550 nm, 600 nm, 650 nm, 700 nm, 750 nm, 800 nm, 850 nm, 900 nm, 1000 nm, etc.). In reference to the sensor arrays described herein, the use of the term sensor element includes the use of a nanoscale sensor element for the sensor element and associated methods.

The term “plurality of sensor elements” refers to more than one, for example, at least two sensor elements. In some embodiments, the plurality of sensor elements includes at least two sensor elements to at least 10¹⁵ sensor elements. In some embodiments, the plurality of sensor elements includes 10⁶-10⁷, 10⁶-10⁸, 10⁶-10⁹, 10⁶-10¹⁰, 10⁶-10¹¹, 10⁶-10¹², 10⁶-10¹³, 10⁶-10¹⁴, 10⁶-10¹⁵, 10⁷-10⁸, 10⁷-10⁹, 10⁷-10¹⁰, 10⁷-10¹¹, 10⁷-10¹², 10⁷-10¹³, 10⁷-10¹⁴, 10⁷-10¹⁵, 10⁸-10⁹, 10⁸-10¹⁰, 10⁸-10¹¹, 10⁸-10¹², 10⁸-10¹³, 10⁸-10¹⁴, 10⁸-10¹⁵, 10⁹-10¹⁰, 10⁹-10¹¹, 10⁹-10¹², 10⁹-10¹³, 10⁹-10¹⁴, 10⁹-10¹⁵, 10¹⁰-10¹¹, 10¹⁰-10¹², 10¹⁰-10¹³, 10¹⁰-10¹⁴, 10¹⁰-10¹⁵, 10¹¹-10¹², 10¹¹-10¹³, 10¹¹-10¹⁴, 10¹¹-10¹⁵, 10¹²-10¹³, 10¹²-10¹⁴, 10¹²-10¹⁵, 10¹³-10¹⁴, 10¹³-10¹⁵, or 10¹⁴-10¹⁵ different sensor elements.

In some embodiments, a plurality of sensor elements comprises a plurality of types of sensor elements. A plurality of sensor elements may comprise at least two to at least 1000 types of sensor elements, alternatively at least two to at least 50 types of sensor elements, alternatively at least 2 to 30 types of sensor elements, alternatively at least 2 to 20 types of sensor elements, alternatively at least 2 to 10 types of sensor elements, alternatively at least 3 to at least 50 types of sensor elements, alternatively at least 3 to at least 30 types of sensor elements, alternatively at least 3 to at least 20 types of sensor elements, alternatively at least 3 to at least 10 types of sensor elements, alternatively at least 4 to at least 50 types of sensor elements, alternatively at least 4 to at least 30 types of sensor elements, alternatively at least 4 to at least 20 types of sensor elements, alternatively at least 4 to at least 10 types of sensor elements, and including any number of types of sensor elements contemplated in between (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, 225, 250, 300, 350, 400, 450, 500, 550, 600, 650, 700, 750, 800, etc.). The plurality of sensor elements may comprise at least 6 types of sensor elements to at least 20 types of sensor elements, or alternatively at least 6 types of sensor elements to at least 10 types of sensor elements. In some embodiments, only one type of sensor element is used in the methods disclosed herein.

The sensor elements may be functionalized to have a wide range of physicochemical properties. Suitable methods of functionalizing the sensor elements are known in the art and depend on composition of the sensor element (e.g. gold, iron oxide, silica, silver, etc.), and include, but are not limited to, for example aminopropyl functionalized, amine functionalized, boronic acid functionalized, carboxylic acid functionalized, methyl functionalized, succinimidyl ester functionalized, PEG functionalized, streptavidin functionalized, methyl ether functionalized, triethoxylpropylaminosilane functionalized, thiol functionalized, PCP functionalized, citrate functionalized, lipoic acid functionalized, BPEI functionalized, carboxyl functionalized, hydroxyl functionalized, and the like. In one embodiment, the sensor elements may be functionalized with an amine group (—NH₂ or a carboxyl group (COOH). In some embodiments, the nanoscale sensor elements are functionalized with a polar functional group. Non-limiting examples of the polar functional group comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group, and the like. In some embodiments, the polar functional group is an ionic functional group. Non-limiting examples of the ionic function group comprise an ammonium group, an imidazolium group, a sulfonium group, a pyridinium group, a pyrrolidinium group, a phosphonium group. In some embodiments, the sensor elements are functionalized with a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

The surface elements may be functionalized to comprise one or more sugar functionalizations. Sugar functionalizations as described herein may comprise any monosaccharide or derivative thereof. Non-limiting example of monosaccharides include glucose, galactose, fructose, glyceraldehyde, erythrose, threose, erythulose, dihydroxyacetone, lyxose, ribulose, xylulose, deoxyribose, altrose, gulose, idose, mannose, talose, psicose, sorbose, tagatose, N-acetylglucosamine, N-acetylgalactosamine, N-acetylneuraminic acid, N-glycolylneuraminic acid, neuraminic acid, 2-keto-3-deoxynononic acid, 3-deoxy-D-manno-2 octulopyranosylonic acid, galacturonic acid, iduronic acid, hamnose, fucose, fuculose, rhamnose, mannoheptulose, sudoheptulose, xylose, ribose, arabinose, glucuronic acid, allose, apiose, ascarylose, and ribitol. Derivatives of monosaccharides may comprise sugar alcohols, amino sugars, uronic acids, ulosonic acids, aldonic acids, aldaric acids, sulfosugars, or any combination or modification thereof. A sugar modification may comprise one or more of acetylation, propylation, formylation, phosphorylation, or sulfonation or addition of one or more of N-acetyl, phosphoethanolamine, inositol, methyl, N-acetyl, O-acetyl, phosphate, phosphocholine, pyruvate, sulfate, sulfide, aminoethylphosphonate, deoxy, carboxylic acid, amine, amide, and ketone. Such modifications may be present at any position on the sugar, as designated by standard sugar naming/notation. In some cases, at least 1, 2, 3, 4, 5, 6, 7, 8, 9, 10 or more modifications are present on the monosaccharide. In some cases, no more than 10, 9, 8, 7, 6, 5, 4, 3, 2, 1, or fewer modifications are present on the monosaccharide. Monosaccharides may comprise any number of carbon atoms. Monosaccharides may comprise any stereoisomer, epimer, enantiomer, or anomer. In some cases, monosaccharides comprise 3, 4, 5, 6, 7, 8, 9, 10, 11, 12, 13, 14, 15, or more carbon atoms. A sugar functionalization may comprise one monosaccharide. A sugar functionalization may comprise a plurality of monosaccharides. A sugar functionalization comprising a plurality of monosaccharides may be referred to as a polysaccharide functionalization. In such cases, the monosaccharides may be connected in any configuration through any suitable glycosidic bond(s). Glycosidic bonds between monosaccharides in a polysaccharide functionalization may be alpha or beta and connect any two carbon atoms between adjacent monosaccharide residues through an oxygen atom.

In some cases, a sugar functionalization comprises a phosphorylated monosaccharide. The phosphorylated monosaccharide may comprise a monosaccharide phosphorylated at any suitable positions. The phosphorylated monosaccharide may comprise a plurality of phosphates. Non-limiting examples of phosphorylated monosaccharide functionalizations comprise dihydroxyacetone phosphate, glyceraldehyde 3-phosphate, glyceraldehyde 2-phosphate, erythrose 2-phosphate, erythrose 3-phosphate, erythrose 4-phosphate, threose 2-phosphate, threose 3-phosphate, threose 4-phosphate, erythulose 1-phosphate, erythulose 3-phosphate, erythulose 4-phosphate, ribose 5-phosphate, ribulose 5-phosphate, xylulose 5-phosphate, glucose 1-phosphate (G1P), glucose 2-phosphate, glucose 3-phosphate, glucose 6-phosphate (G6P), fructose 1-phosphate, fructose 2-phosphate, fructose 3-phosphate, fructose 6-phosphate (F6P), fructose 1,6-bisphosphate, fructose 2,6-bisphosphate, and sedoheptulose 7-phosphate. In some embodiments, the sugar functionalization comprises a glucose phosphate. In some embodiments, the sugar functionalization comprises a glucose-6-phosphate.

The physicochemical properties of the sensor elements may be modified by modification of the surface charge. For example, the surface can be modified to provide a net neutral charge, a net positive surface charge, a net negative surface charge, or a zwitterionic charge. The charge of the surface can be controlled either during synthesis of the element or by post-synthesis modification of the charge through surface functionalization. For polymeric sensor elements (e.g., polymeric particles), differences in charge can be obtained during synthesis by using different synthesis procedures, different charged comonomers, and in inorganic substances by having mixed oxidation states.

Sensor elements (e.g., surfaces) as described herein can have a ranged of different zeta potentials. Surfaces can have a zeta potential which is positive or negative in charge. In some cases, surfaces have a zeta potential with a magnitude of 10 mV to 60 mV. In some cases, surfaces have a zeta potential with a magnitude of 10 mV to 20 mV, 10 mV to 30 mV, 10 mV to 40 mV, 10 mV to 50 mV, 10 mV to 60 mV, 20 mV to 30 mV, 20 mV to 40 mV, 20 mV to 50 mV, 20 mV to 60 mV, 30 mV to 40 mV, 30 mV to 50 mV, 30 mV to 60 mV, 40 mV to 50 mV, 40 mV to 60 mV, or 50 mV to 60 mV. In some cases, surfaces have a zeta potential with a magnitude of 10 mV, 20 mV, 30 mV, 40 mV, 50 mV, or 60 mV. In some cases, surfaces have a zeta potential with a magnitude of at least 10 mV, 20 mV, 30 mV, 40 mV, or 50 mV. In some cases, surfaces have a zeta potential with a magnitude of at most 20 mV, 30 mV, 40 mV, 50 mV, or 60 mV.

The method may, in some embodiments, use a plurality of sensor elements. Non-limiting examples of the plurality of sensor elements include, but are not limited to, (a) a plurality of sensor elements made of the same material but differing in physiochemical properties, (b) a plurality of sensor elements where one or more sensor element is made of a different material with the same or differing physiochemical properties, (c) a plurality of sensor elements made of the same material differing in size, (d) a plurality of sensor elements made of different material with relatively the same size; (e) a plurality of sensor elements made of different material and made of different sizes, (f) a plurality of sensor elements in which each element is made of a different material, (g) a plurality of sensor elements having different charges, among others. The plurality of sensor elements can be in any suitable combination of two or more sensor elements in which each sensor element provides a unique biomolecule corona signature. For example, the plurality of sensor elements may include one or more liposomes and one or more particles described herein. In one embodiment, the plurality of sensor elements can be a plurality of liposomes with varying lipid content and/or varying charges (cationic/anionic/neutral). In another embodiment, the plurality of sensors may contain one or more nanoparticles made of the same material but of varying sizes and physiochemical properties. In another embodiment, the plurality of sensors may contain one or more particles made of differing materials (e.g., silica and polystyrene) with similar or varying sizes and/or physiochemical properties (e.g., modifications, for example, —NH₂, —COOH functionalization). These combinations are purely provided as examples and are non-limiting to the scope of the disclosure.

A sensor element may comprise a particle, such as a nanoparticle or a microparticle. A sensor element may be a particle, such as a nanoparticle or a microparticle. A sensor element may comprise a surface or a portion of a surface of a material. A sensor element may comprise a porous material (e.g., a polymer matrix) into which biomolecules can intercalate. A sensor element may comprise a material with projections, such as polymers, oligomers, or metal dendrites. A sensor element may comprise an aggregate of particles, such as a nanoworm.

### Particle Materials

The particles used in the methods disclosed herein can be made of a variety of different materials. A plurality of particles can comprise specific types of nanoparticles to identify a broad range of proteins in the sample, or to selectively assay for a particular protein or set of proteins of interest.

The particles may comprise at least 1 particle distinct type, at least 2 distinct particle types, at least 3 distinct particle types, at least 4 distinct particle types, at least 5 distinct particle types, at least 6 distinct particle types, at least 7 distinct particle types, at least 8 distinct particle types, at least 9 distinct particle types, at least 10 distinct particle types, at least 11 distinct particle types, at least 12 distinct particle types, at least 13 distinct particle types, at least 14 distinct particle types, at least 15 distinct particle types, at least 16 distinct particle types, at least 17 distinct particle types, at least 18 distinct particle types, at least 19 distinct particle types, at least 20 distinct particle types, at least 25 distinct particle types, at least 30 distinct particle types, at least 35 distinct particle types, at least 40 distinct particle types, at least 45 distinct particle types, at least 50 distinct particle types, at least 55 distinct particle types, at least 60 distinct particle types, at least 65 distinct particle types, at least 70 distinct particle types, at least 75 distinct particle types, at least 80 distinct particle types, at least 85 distinct particle types, at least 90 distinct particle types, at least 95 distinct particle types, at least 100 distinct particle types, from 1 to 5 distinct particle types, from 5 to 10 distinct particle types, from 10 to 15 distinct particle types, from 15 to 20 distinct particle types, from 20 to 25 distinct particle types, from 25 to 30 distinct particle types, from 30 to 35 distinct particle types, from 35 to 40 distinct particle types, from 40 to 45 distinct particle types, from 45 to 50 distinct particle types, from 50 to 55 distinct particle types, from 55 to 60 distinct particle types, from 60 to 65 distinct particle types, from 65 to 70 distinct particle types, from 70 to 75 distinct particle types, from 75 to 80 distinct particle types, from 80 to 85 distinct particle types, from 85 to 90 distinct particle types, from 90 to 95 distinct particle types, from 95 to 100 distinct particle types, from 1 to 100 distinct particle types, from 20 to 40 distinct particle types, from 5 to 10 distinct particle types, from 3 to 7 distinct particle types, from 2 to 10 distinct particle types, from 6 to 15 distinct particle types, or from 10 to 20 distinct particle types. A plurality of particles may comprise from 3 to 10 particle types. A plurality of particles may comprise from 4 to 11 distinct particle types. A plurality of particles may comprise from 5 to 15 distinct particle types. A plurality of particles may comprise from 5 to 15 distinct particle types. A plurality of particles may comprise from 8 to 12 distinct particle types. A plurality of particles may comprise from 9 to 13 distinct particle types. A plurality of particles may comprise 10 distinct particle types. The particle types may include nanoparticles.

For example, in the methods of the present disclosure a plurality of particles having at least 2 distinct particle types, at least 3 different surface chemistries, at least 4 different surface chemistries, at least 5 different surface chemistries, at least 6 different surface chemistries, at least 7 different surface chemistries, at least 8 different surface chemistries, at least 9 different surface chemistries, at least 10 different surface chemistries, at least 11 different surface chemistries, at least 12 different surface chemistries, at least 13 different surface chemistries, at least 14 different surface chemistries, at least 15 different surface chemistries, at least 20 different surface chemistries, at least 25 different surface chemistries, at least 30 different surface chemistries, at least 35 different surface chemistries, at least 40 different surface chemistries, at least 45 different surface chemistries, at least 50 different surface chemistries, at least 100 different surface chemistries, at least 150 different surface chemistries, at least 200 different surface chemistries, at least 250 different surface chemistries, at least 300 different surface chemistries, at least 350 different surface chemistries, at least 400 different surface chemistries, at least 450 different surface chemistries, at least 500 different surface chemistries, from 2 to 500 different surface chemistries, from 2 to 5 different surface chemistries, from 5 to 10 different surface chemistries, from 10 to 15 different surface chemistries, from 15 to 20 different surface chemistries, from 20 to 40 different surface chemistries, from 40 to 60 different surface chemistries, from 60 to 80 different surface chemistries, from 80 to 100 different surface chemistries, from 100 to 500 different surface chemistries, from 4 to 15 different surface chemistries, or from 2 to 20 different surface chemistries.

The methods of the present disclosure provides a plurality of particles having at least 2 different physical properties, at least 3 different physical properties, at least 4 different physical properties, at least 5 different physical properties, at least 6 different physical properties, at least 7 different physical properties, at least 8 different physical properties, at least 9 different physical properties, at least 10 different physical properties, at least 11 different physical properties, at least 12 different physical properties, at least 13 different physical properties, at least 14 different physical properties, at least 15 different physical properties, at least 20 different physical properties, at least 25 different physical properties, at least 30 different physical properties, at least 35 different physical properties, at least 40 different physical properties, at least 45 different physical properties, at least 50 different physical properties, at least 100 different physical properties, at least 150 different physical properties, at least 200 different physical properties, at least 250 different physical properties, at least 300 different physical properties, at least 350 different physical properties, at least 400 different physical properties, at least 450 different physical properties, at least 500 different physical properties, from 2 to 500 different physical properties, from 2 to 5 different physical properties, from 5 to 10 different physical properties, from 10 to 15 different physical properties, from 15 to 20 different physical properties, from 20 to 40 different physical properties, from 40 to 60 different physical properties, from 60 to 80 different physical properties, from 80 to 100 different physical properties, from 100 to 500 different physical properties, from 4 to 15 different physical properties, or from 2 to 20 different physical properties.

Particles used in the methods of the present disclosure can be made from various materials. For example, nanoparticle materials consistent with the present disclosure include metals, polymers, magnetic materials, and lipids. Magnetic nanoparticles may be iron oxide nanoparticles. Examples of metal materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron and cadmium, or any other material described in U.S. Pat. No. 7,749,299.

Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). In some embodiments, the polymer is a lipid-terminated polyalkylene glycol and a polyester, or any other material disclosed in U.S. Pat. No. 9,549,901. A polymer may also be a liposome.

Examples of lipids that can be used to form the nanoparticles of the present disclosure include cationic, anionic, and neutrally charged lipids. For example, nanoparticles can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol, or any other material listed in U.S. Pat. No. 9,445,994.

In various cases, the core of the nanoparticles can include an organic particle, an inorganic particle, or a particle including both organic and inorganic materials. For example, the particles can have a core structure that is or includes a metal particle, a quantum dot particle, a metal oxide particle, or a core-shell particle. For example, the core structure can be or include a polymeric particle or a lipid-based particle, and the linkers can include a lipid, a surfactant, a polymer, a hydrocarbon chain, or an amphiphilic polymer. For example, the linkers can include polyethylene glycol or polyalkylene glycol, e.g., the first ends of the linkers can include a lipid bound to polyethylene glycol (PEG) and the second ends can include functional groups bound to the PEG. A particle may have a core-shell structure. In some cases, a particle has a core comprising a first material or composite and a plurality of shells comprising different materials or composites. In some cases, a particle has a magnetic core surrounded by a non-magnetic or plurality of non-magnetic shells. For example, a particle may comprise a magnetic iron oxide core surrounded by a non-magnetic polymer shell. In some cases, magnetic core has a 10 nm to 500 nm diameter, and the shell has a 5 nm to 100 nm thickness.

### Properties of Particles

Nanoparticles that are consistent with the present disclosure can be made and used in methods of forming protein coronas after incubation in a biofluid at a wide range of sizes. For example, the nanoparticles disclosed herein can be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm.

Additionally, particles can have a homogenous size distribution or a heterogeneous size distribution. Polydispersity index (PDI), which can be measured by techniques such as dynamic light scattering, is a measure of the size distribution. A low PDI indicates a more homogeneous size distribution and a higher PDI indicates a more heterogeneous size distribution. In some cases, a plurality of particles has a PDI of 0.01 to 0.1, 0.1 to 0.5, 0.5 to 1, 1 to 5, 5 to 20, or greater than 20.

Particles disclosed herein can have a range of different surface charges. Particles can be negatively charged, positively charged, or neutral in charge. In some embodiments, particles have a surface charge of −500 mV to −450 mV, −450 mV to −400 mV, −400 mV to −350 mV, −350 mV to −300 mV, −300 mV to −250 mV, −250 mV to −200 mV, −200 mV to −150 mV, −150 mV to −100 mV, −100 mV to −90 mV, −90 mV to −80 mV, −80 mV to −70 mV, −70 mV to −60 mV, −60 mV to −50 mV, −50 mV to −40 mV, −40 mV to −30 mV, −30 mV to −20 mV, −20 mV to −10 mV, −10 mV to 0 mV, 0 mV to 10 mV, 10 mV to 20 mV, 20 mV to 30 mV, 30 mV to 40 mV, 40 mV to 50 mV, 50 mV to 60 mV, 60 mV to 70 mV, 70 mV to 80 mV, 80 mV to 90 mV, 90 mV to 100 mV, 100 mV to 110 mV, 110 mV to 120 mV, 120 mV to 130 mV, 130 mV to 140 mV, 140 mV to 150 mV, 150 mV to 200 mV, 200 mV to 250 mV, 250 mV to 300 mV, 300 mV to 350 mV, 350 mV to 400 mV, 400 mV to 450 mV, 450 mV to 500 mV, −500 mv to −400 mV, −400 mv to −300 mV, −300 mv to −200 mV, −200 mv to −100 mV, −100 mv to 0 mV, 0 mv to 100 mV, 100 mv to 200 mV, 200 mv to 300 mV, 300 mv to 400 mV, or 400 mv to 500 mV.

Various particle morphologies are consistent with the particle types in panels of the present disclosure. For example, particles may be spherical, colloidal, square shaped, rods, wires, cones, pyramids, or oblong.

### Biomolecule Coronas

Provided herein are methods capable of generating biomolecule coronas comprising one or more sensor elements wherein the sensor elements differ from each other in at least one physicochemical property. The sensor elements may comprise a plurality of particles (e.g., nanoparticles). The sensor elements may be a plurality of particles. The sensor elements may be able to bind a plurality of biomolecules in a complex biological sample to produce a biomolecule corona signature. The sensor elements may comprise a plurality of distinct biomolecule corona signatures.

A biomolecule of interest (e.g., IGF-1) may be enriched in a biomolecule corona relative to the untreated sample (e.g., a sample that is not assayed using particles). The biomolecule of interest may be a protein. The biomolecule corona may be a protein corona. A level of enrichment may be the percent increase or fold increase in relative abundance of the biomolecule of interest (e.g., number of copies of the biomolecule of interest versus the total number of biomolecules) in the biomolecule corona as compared to the biological sample from which the biomolecule corona was collected. A biomolecule of interest may be enriched in a biomolecule corona by increasing the abundance of the biomolecule of interest in the biomolecule corona as compared to the sample that has not been contacted to the sensor element. A biomolecule of interest may be enriched by decreasing the abundance of a biomolecule that is in high abundance biological sample.

In some embodiments, the methods of the present application may be used to detect the analyte proteins in at least 20 samples per day using a single mass spectrometer. In some embodiments, the methods of the present application may be used to detect the analyte proteins in at least 50 samples per day using a single mass spectrometer. In some embodiments, the methods of the present application may be used to detect the analyte proteins in at least 100 samples per day using a single mass spectrometer. In some embodiments, the methods of the present application may be used to detect the analyte proteins in at least 120 samples per day using a single mass spectrometer. In some embodiments, the methods of the present application may be used to detect the analyte proteins in at least 250 samples per day using a single mass spectrometer.

### Identifying Analyte Proteins

The methods disclosed herein can be used to identify or quantify specific biomarkers. and/or modified forms (e.g., post-translationally modified proteoforms) or derivatives thereof (e.g., peptides). “Feature intensities,” as disclosed herein, generally refers to the intensity of a signal from an analytical measurement, for example the intensity of a mass to charge ratio from a mass spectrometry run of a sample. Using the data analysis methods described herein, feature intensities of peptides and peptide fragments can be sorted into protein groups. Protein groups refer to two or more proteins that are identified by a shared peptide sequence. Alternatively, a protein group can refer to one protein that is identified using a unique identifying amino acid sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). Alternatively, if the peptide sequence is unique to a single protein (Protein 1), a protein group could be the “ZZX” protein group having one member (Protein 1). Each protein group can be supported by more than one peptide sequence. Proteins detected, identified, or quantified according to the instant disclosure can refer to a distinct protein detected in the sample (e.g., distinct relative other proteins detected using mass spectrometry). Thus, analysis of proteins present in distinct coronas corresponding to the distinct sensor element types yields a high number of feature intensities. This number decreases as feature intensities are processed into distinct peptides, further decreases as distinct peptides are processed into distinct proteins, and further decreases as peptides are grouped into protein groups (two or more proteins that share a distinct peptide sequence).

### Detection of Analyte Proteins

As described elsewhere herein, the present disclosure provides methods for detecting or identifying a presence or absence of one or more of substances (e.g., analyte proteins, such as one or more metabolic biomarkers or their modified form as described herein) in a sample using a targeted method. The presence or absence of the one or more of substances in the sample may be indicative of a likelihood of a sample being positive for a disease or condition. Non-limiting examples of detectors for use with the methods described herein may include targeted mass spectrometry (MS) (e.g., quadrupole MS, orthogonal MS, etc.), affinity-based detection methods, or combinations thereof.

One example of a targeted mass spectrometry technique is multiple reaction monitoring (MRM) or selective reaction monitoring (SRM). Another example of targeted mass spectrometry is parallel reaction monitoring (PRM). During a typical SRM or PRM run, a mass spectrometer continuously acquires spectra at an expected mass to charge (m/z) ratio and chromatographic retention time (RT) of target analytes (e.g., peptides). SRM experiments are performed on triple quadrupole mass spectrometers where precursor ions are selected in the first quadrupole and fragmented in the second quadrupole. Target-specific fragment ions are then selected in the third quadrupole for detection. PRM experiments are performed on systems able to record whole fragment spectra, such as quadrupole-orbitrap type mass spectrometers and QqTOF systems. In PRM, all fragment ions of a selected precursor are measured in parallel. An inclusion list is passed on to the mass spectrometer that dictates the precursor m/z ratio windows and RT windows. On specific systems more scan parameters can be included such as scan specific fragmentation energies or injection times. For SRM, the inclusion list additionally specifies the m/z of the fragment ions to be monitored. Targeted mass spectrometric assays may allow for particularly sensitive and specific identification and/or quantification of target analytes.

In some cases, methods of the present disclosure comprise an operation of assaying proteins in a protein corona with a targeted assay. In some cases, the target assay comprises a targeted MS method. In some cases, the targeted MS method is SRM, MRM, or PRM. In some cases, the targeted MS method comprises liquid chromatography-tandem mass spectrometry (LC-MS/MS).

In some cases, identification and/or quantification of analyte proteins may be done by affinity assays. Affinity assay as described herein may include, but are not limited to, immunoassays, enzyme-linked immunosorbent assays (ELISA), radioimmunoassays (RIA), ligand binding assays, aptamer biding assays, functional assays, enzymatic assays, immunoprecipitations (IP), chromatography, enrichments, pull-downs, and the like. In some cases, the methods as described herein may not comprise an affinity-based assay.

### Disease Detection or Screening

A variety of metabolic diseases, conditions, or disorders may be studied, diagnosed, and/or monitored by the methods of the present disclosure. Disease study, diagnosis and/or monitoring can include detecting one or more analyte proteins (e.g., biomarkers) that are indicative of the disease in a sample. In some cases, the sample is derived from a subject. Additionally, the one or more biomarkers may be quantified and used to determine the likelihood that an individual has or diagnose an individual with a disease or condition.

The methods of the present disclosure can be used to detect, identify a risk of a wide range of disease states in a given sample. For example, the systems and methods of the present disclosure can be used to detect, among other, obesity, diabetes, and polycystic ovary syndrome (PCOS).

In some cases, the methods of the disclosure can be used to determine the disease state of a subject, diagnose or prognose a disease in a subject or identify unique patterns of analyte proteins (e.g., biomarkers) that are associated with a disease state or a disease or disorder. For example, the changes in the analyte proteins detected in a sample from a subject over time (e.g., days, months, years) allows for the ability to track a disease or disorder in a subject (e.g., disease state) which may be broadly applicable to determination of the presence or absence of analyte proteins or particular amounts thereof that can be associated with the early stage of a disease or any other disease state.

In some cases, the disease state or condition comprises a metabolic disorder. The metabolic disorder may be a congenital disorder, or it may be an acquired disorder. Examples of metabolic disorders include, but are not limited to, diabetes, diabetic neuropathy, insulin resistance, goiter, metabolic syndrome, obesity, hyperlipidemia, dyslipidemia, hypertriglyceridemia, high cholesterol (hypercholesterolemia), hyperglycemia, glucose intolerance, arteriosclerosis, hypertension, non-alcoholic steatohepatitis (NASH), non-alcoholic fatty liver, non-alcoholic fatty liver disease (NAFLD), hepatic steatosis, amino acidemia, maple syrup urine disease (MSUD), homocystinuria, 3-hydroxy-3-methylglutaryl-CoA lyase deficiency, 3-methylcrotonyl-CoA carboxylase deficiency, 3-methylglutaconyl-CoA hydratase deficiency, alkaptonuria, aminoacylase 1 deficiency, arginase deficiency, arginine: glycine amidinotransferase deficiency, arginosuccinic aciduria, asparagine synthetase deficiency, beta-ketothiolase deficiency, dihydrolipoamide dehydrogenase deficiency, glutamate formiminotransferase deficiency, glutaric acidemia, guanidinoacetate methyltransferase deficiency, Hartnup disease, histidinemia, hyperlysinemia, hypermethioninemia, hyperprolinemia, isobutyryl-CoA dehydrogenase deficiency, isolate sulfite oxidase deficiency, isovaleric acidemia, Lesch-Nyhan syndrome, methylmalonic acidemia, nonketotic hyperglycinemia, phosphoglycerate dehydrogenase deficiency, primary hyperoxaluria, prolidase deficiency, short/branched chain acyl-CoA dehydrogenase deficiency, tyrosinemia, organic acidemia, propionic acidemia, phenylketonuria (PKU), Barth syndrome (3-methylglutaconic aciduria), glutaric aciduria, 2-hydroxoglutaric aciduria, fatty acid oxidation disorders (FAODs), medium-chain acyl-CoA dehydrogenase deficiency, very long-chain acyl-CoA dehydrogenase deficiency (VLCAD), 3-hydroxyacyl-CoA dehydrogenase deficiency, trifunctional protein deficiency, carnitine deficiency, carnitine palmitoyltransferase deficiency, carnitine-acylcarnitine translocase deficiency, carnitine transporter deficiency, short-chain acyl-CoA dehydrogenase deficiency (SCADD), multiple acyl-CoA dehydrogenase deficiency, lipid storage disorders, Gml gangliosidosis, Tay-Sachs disease, Sandhorff disease, Fabry disease, Gaucher disease, Niemann-Prick disease, Krabbe disease, cerebrotendinous xanthomatosis, Chanarin-Dorfman syndrome, hypobetalipoproteinemia, lipoprotein lipase deficiency, Farber lipogranulomatosis, lysosomal acid lipase deficiency, Smith-Lemli-Optiz syndrome, lactic acidosis, mitochondrial encephalopathy, mitochondrial myopathy, mitochondrial recessive ataxia syndrome, muscular dystrophies, myoclonic epilepsy, uneven red fiber disease, gastrointestinal myoneurogenic encephalopathy, retinitis pigmentosa, Pearson syndrome, primary coenzyme Q10 deficiency, mitochondrial complex I deficiency, mitochondrial complex II deficiency, mitochondrial complex II deficiency, cytochrome c oxidase deficiency, mucolipidoses, mucopolysaccharidoses, and Zellweger syndrome.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1: Identification and Quantification of Metabolic Biomarkers from Plasma

Plasma samples from four subjects were obtained for this study. The four subjects comprised two males (ages 13 and 21) and two females (ages 41 and 59). The plasma samples were incubated with five functionalized nanoparticles, and the resulting protein coronas were interrogated using targeted liquid chromatography-tandem mass spectrometry (LC-MS/MS) analysis.

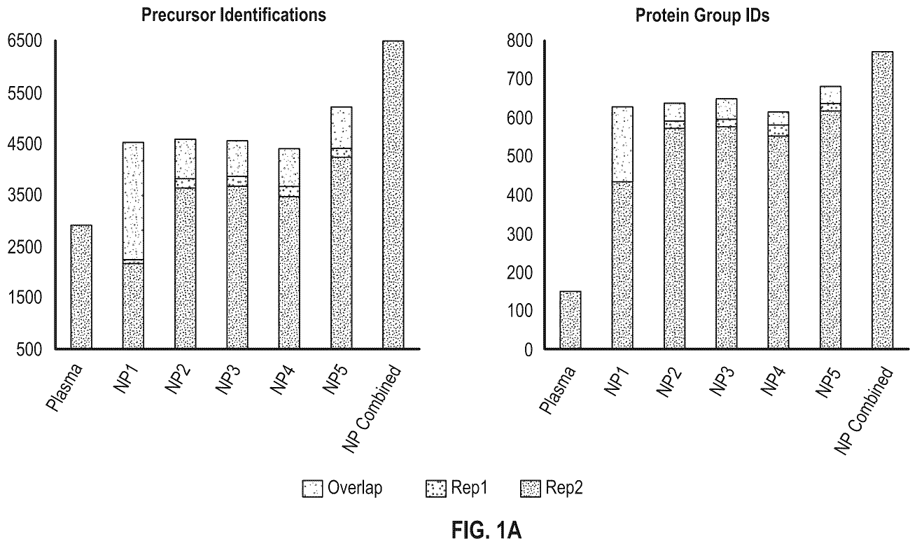

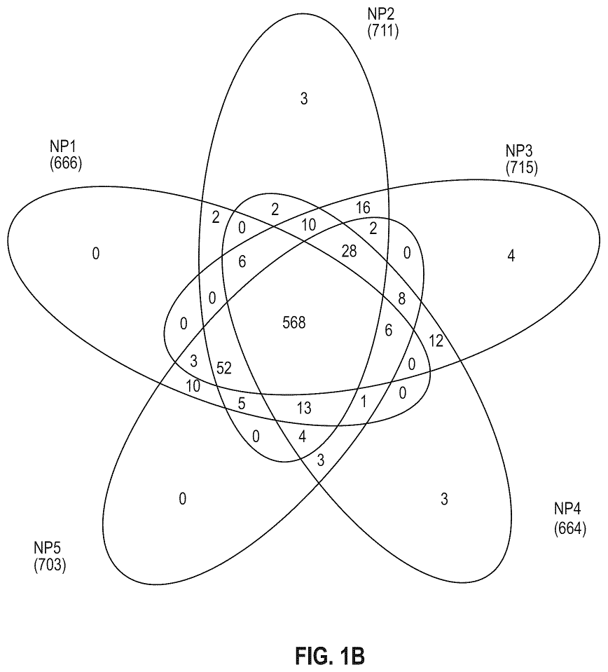

FIG. 1A illustrates the identified proteins (left panel) and precursor molecules (e.g., peptides) (right panel) detected from the protein coronas on each nanoparticle. FIG. 1B depicts a Venn diagram showing counts of which proteins were identified in the corona(s) of corresponding particles.

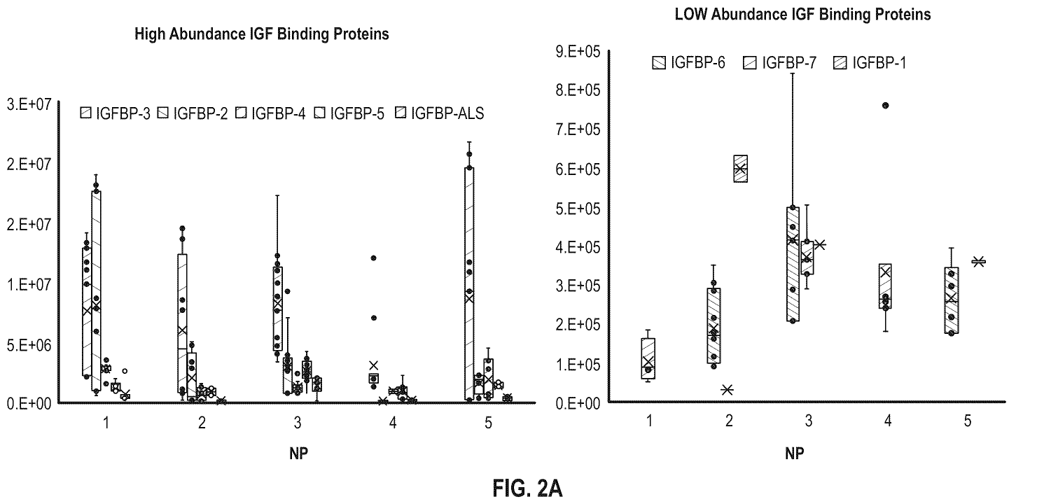

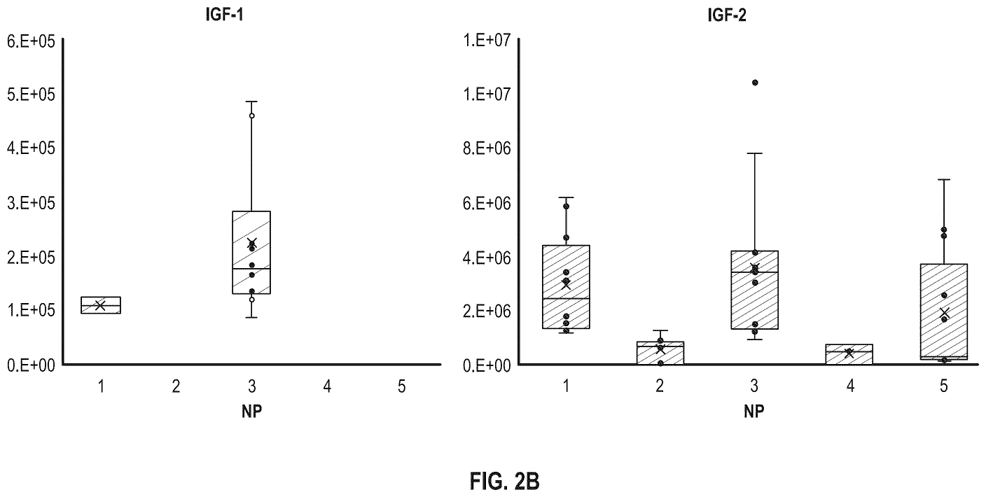

FIG. 2A is a graph showing the distribution of relative insulin-like growth factor 1 (IGF-1), IGF-2, insulin-like growth factor acid labile subunit (IGFALS), and insulin-like growth factor binding proteins 1 through 7 (IGFBP1-7) abundance (reported as log intensity) across each nanoparticle type (NP1-NP5).

FIG. 2B depicts graphs showing the distribution of relative amounts of IGF-1 (left panel) and IGF-2 (right panel) across nanoparticle types. As shown in FIG. 2B, NP3, which comprises a glucose 6-phosphate functionalized surface, is able to detect both IGF-1 and IGF-2 across all tested samples at varying concentrations.

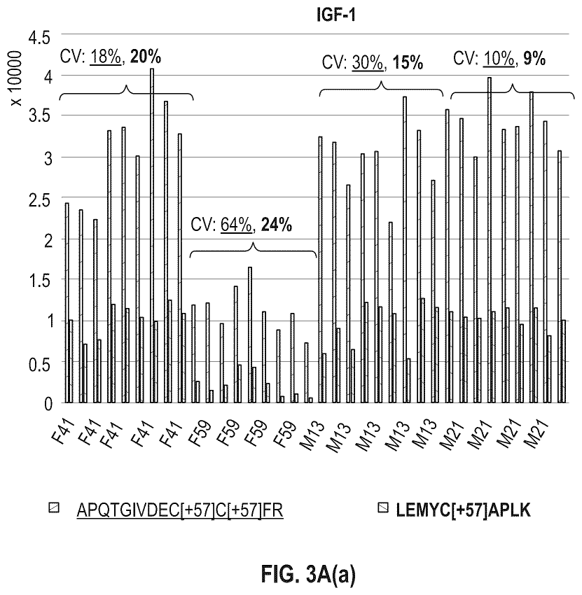

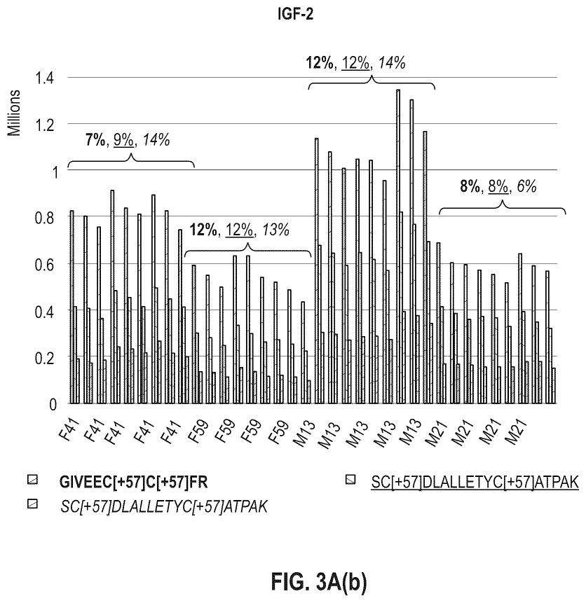

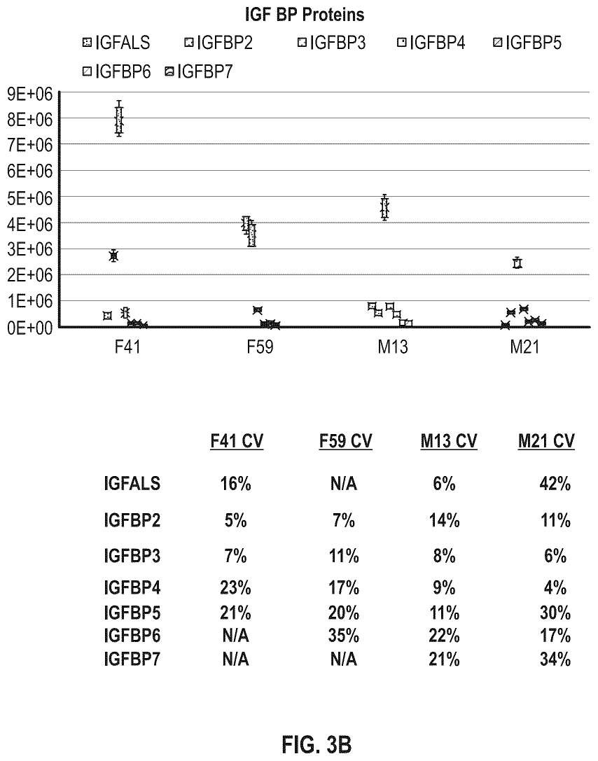

FIG. 3A shows calculated coefficients of variation (CVs) for IGF-1 (a) and IGF-2 (b) derived peptides across all replicates analyzed. In general, CVs are low, ranging from 9%-24%, 7%-12%, 8%-13%, and 6%-14%, indicating generally low technical variability. One plasma sample (F59) showed relatively high CVs across all peptides, including 64% and 24% for IGF-derived peptides, as shown in the left panel of FIG. 3A, suggesting the sample may have been degraded. FIG. 3B shows calculated CVs for IGFBP2-7 and IGFALS across all replicates tested. Again, CVs were generally low (5%-25% or less), with a few outlier values.

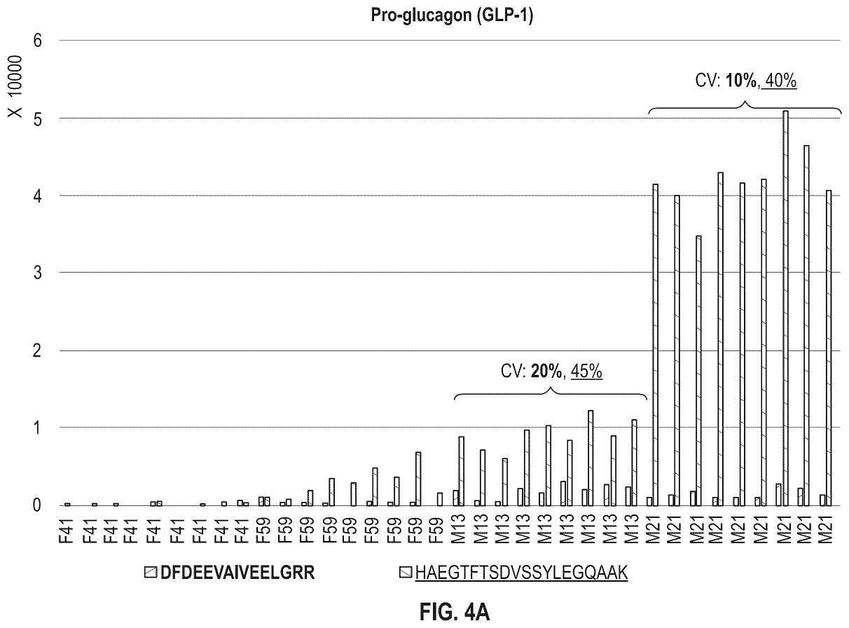

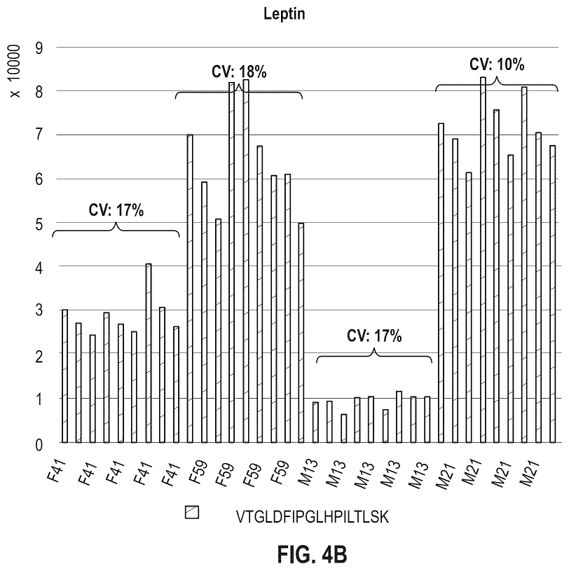

FIGS. 4A and 4B show calculated coefficients of variation (CVs) for proglucagon (a progenitor of glucagon-like peptide 1 (GLP-1)) (FIG. 4A) and leptin (FIG. 4B) derived peptides across all replicates analyzed. In general, CVs are low, ranging from about 10%-20%, indicating generally low technical variability. CVs for the second proglucagon peptide were relatively high (45% and 40%) due to the low quantities detected, suggesting that GLP-1 and proglucagon can be differentially identified and quantified by the methods described herein

### Example 2: Quantification of Insulin-Like Growth Factor 2 (IGF2) from Plasma Using Data Independent Acquisition (DIA)-Liquid Chromatograph-Mass Spectrometry (DIA LC-MS)

Human and bovine plasma samples were mixed at seven different volume ratios and incubated with PDMAPMA-coated magnetic nanoparticles to form protein coronas. The protein coronas were washed, digested with trypsin, and prepared for liquid chromatography mass spectrometry analysis. DIA LC-MS was performed, and the intensity of peaks associated with bovine IGF2 was determined. The same mixtures of neat plasma were also analyzed by DIA LC-MS. Each mixture was processed in four replicates. Table 3 below shows the intensity data.

When analyzing bovine IGF2 from the protein corona, the intensity increased linearly with the fraction of bovine plasma in the sample. The limit of detection (LOD) was less than about 10% of the abundance for endogenous levels of bovine IGF2. These results indicate that protein corona may be used to quantify relevant analyte proteins from plasma using mass spectrometry.

TABLE 3
Measured Intensities for Bovine IGF2

| Bovine | Corona | Neat |
|---|---|---|
| Fraction | Intensity | Intensity |
| 0 | 2683180 | 640854 |
| 0 | 2757330 | 476077 |
| 0 | 1988490 | 588037 |
| 0 | 2467470 | 432923 |
| 0.01 | 2855700 | 676500 |
| 0.01 | 2694750 | 655667 |
| 0.01 | 2920170 | 748727 |
| 0.01 | 3715890 | 787198 |
| 0.1 | 4014990 | 1043520 |
| 0.1 | 3528390 | 1020170 |
| 0.1 | 3908320 | 974698 |
| 0.1 | 4070430 | 893436 |
| 0.333333 | 5689290 | 901417 |
| 0.333333 | 7205840 | 863383 |
| 0.333333 | 6830210 | 915938 |
| 0.333333 | 5385460 | 875900 |
| 0.4 | 6479570 | 875946 |
| 0.4 | 7022500 | 1011640 |
| 0.4 | 6727490 | 939506 |
| 0.4 | 6400860 | 940127 |
| 0.5 | 6028760 | 715856 |
| 0.5 | 6332940 | 0 |
| 0.5 | 6212920 | 987000 |
| 0.5 | 7258980 | 874913 |
| 1 | 14727500 | 1798420 |
| 1 | 13113100 | 1377750 |
| 1 | 16738800 | 1688840 |
| 1 | 16942200 | 1545540 |

What is claimed is:

1. A method of analyzing a biological sample, comprising:
(a) incubating said biological sample with a surface to form a protein corona; and
(b) assaying proteins in the protein corona using a targeted method to determine a presence or absence of one or more analyte proteins, wherein said one or more analyte proteins is selected from insulin-like growth factor I (IGF-1), insulin-like growth factor II (IGF-2), IGF binding protein 1 (IGFBP1), IGF binding protein 2 (IGFBP2), IGF binding protein 3 (IGFBP3), IGF binding protein 4 (IGFBP4), IGF binding protein 5 (IGFBP5), IGF binding protein 6 (IGFBP6), IGF binding protein 7 (IGFBP7), Insulin-like growth factor binding protein acid labile subunit (IGFALS), glucagon-like peptide 1 (GLP-1), glucagon-like peptide 2 (GLP-2), leptin, and any combination thereof.

2. The method of claim 1, wherein said surface comprises a surface functionalization.

3. The method of claim 2, wherein said surface functionalization comprises an amine functionalization.

4. The method of claim 3, wherein said surface functionalization comprises a dimethyl amino functionalization.

5. The method of any one of claims 1-4, wherein said surface functionalization comprises a sugar.

6. The method of claim 5, wherein said sugar comprises a sugar phosphate.

7. The method of claim 5 or 6, wherein said sugar comprises glucose.

8. The method of claim 5, wherein said sugar comprises glucose 6-phosphate.

9. The method of any one of claims 5-8, wherein said sugar is a monosaccharide.

10. The method of any one of claims 1-9, wherein said surface is a surface of a particle.

11. The method of claim 10, wherein said particle is a nanoparticle.

12. The method of claim 10, wherein said particle is a microparticle.

13. The method of claim 10, wherein said particle has an average diameter of less than 500 nm.

14. The method of any one of claims 10-13, wherein said particle comprises a magnetic material.

15. The method of claim 14, wherein said particle is a superparamagnetic iron oxide particle.

16. The method of any one of claims 10-15, wherein said particle comprises an iron oxide material.

17. The method of claim 16, wherein said particle comprises an iron oxide core.

18. The method of claim 16, wherein said particle comprises an iron oxide particle embedded in a polystyrene core.

19. The method of any one of claims 10-18, wherein said particle comprises a polymer coating.

20. The method of claim 19, wherein said polymer coating comprises said surface functionalization.

21. The method of claim 19 or 20, wherein said particle comprises a polymethacrylamide or polyacrylamide coating comprising an amine functional group.

22. The method of claim 19 or 20, wherein said particle comprises a poly(N-(3-(dimethylamino) propyl) methacrylamide) (PDMAPMA) coating.

23. The method of any one of claims 10-22, wherein said particle comprises a positive zeta potential.

24. The method of claim 23, wherein said zeta potential has a magnitude greater than about 10 millivolts (mV) or great than about 20 mV.

25. The method of claim 23 or 24, wherein said zeta potential has a magnitude less than about 40 mV or about 40 mV.

26. The method of claim 23, wherein said zeta potential has a magnitude from about 20 mV to about 40 mV.

27. The method of any one of claims 10-21, wherein said particle comprises a negative zeta potential.

28. The method of claim 27, wherein said zeta potential has a magnitude greater than about 10 millivolts or about 20 (mV).

29. The method of claim 27 or 28, wherein said zeta potential has a magnitude less than about 50 mV or about 40 mV.

30. The method of any one of claims 1-29, wherein said surface is comprised of a plurality of surfaces, and wherein (a) comprises contacting said biological sample with said plurality of surfaces.

31. The method of claim 30, wherein said plurality of surfaces have the same surface functionalization.

32. The method of claim 30, wherein at least 90% by area of said plurality of surfaces have the same surface functionalization.

33. The method of claim 30, wherein at least 97% by area of said plurality of surfaces have the same surface functionalization.

34. The method of any one of claims 30-33, wherein said plurality of surfaces is a plurality of surfaces of particles.

35. The method of claim 34, wherein said particles are characterized by a polydispersity index (PDI) less than about 0.1.

36. The method of claim 34 or 35, wherein a second surface of said plurality of surfaces comprises a second surface functionalization.

37. The method of claim 34 or 35, wherein each surface of said plurality of surfaces comprises said surface functionalization.

38. The method of any one of claims 1-37, wherein said assaying comprises quantifying an amount of said one or more analyte proteins.

39. The method of any one of claims 1-38, wherein said assaying comprises targeted mass spectrometry.

40. The method of claim 39, wherein said targeted mass spectrometry comprises tandem mass spectrometry (MS/MS).

41. The method of claim 40, wherein said assaying comprises liquid chromatography-tandem mass spectrometry (LC-MS/MS).

42. The method of any one of claims 39-41, wherein said assaying comprises multiple reaction monitoring (MRM), parallel reaction monitoring (PRM), or selected reaction monitoring (SRM).

43. The method of claim any one of claim 39-42, wherein the analyte proteins are detected using a single mass spectrometry injection.

44. The method of any one of claims 1-43, wherein said assaying comprises affinity-based detection.

45. The method of claim 44, wherein said assaying comprises peptide or aptamer binding.

46. The method of any one of claims 1-45, further comprising, prior to said assaying said analyte proteins, separating at least a portion of said analyte proteins from said surface.

47. The method of any one of claims 1-46, further comprising, digesting at least a portion of said biomolecules adsorbed to said surface.

48. The method of claim 47, wherein said digesting comprises digesting said at least a portion of said biomolecules with a protease (e.g., trypsin).

49. The method of any one of claims 1-48, wherein said assaying comprises detecting or quantifying one or more peptides derived from said one or more analyte proteins.

50. The method of claim 49, wherein said one or more peptides are selected from the peptides in Table 1 or Table 2.

51. The method of any one of claims 1-50, wherein said biological sample is blood, plasma, or serum.

52. The method of any one of claims 1-51, wherein said assaying comprises detecting or quantifying IGF1 and IGF2.

53. The method of any one of claims 1-52, wherein said assaying comprises detecting or quantifying GLP-1 and leptin.

54. The method of any one of claims 1-53, wherein said assaying comprises detecting or quantifying at least one of IGFBP1, IGFBP2, IGFBP3, IGFBP4, IGFBP5, IGFBP6, or IGFBP7, or a derivative thereof.

55. The method of any one of claims 1-53, wherein said assaying comprises detecting or quantifying at least two IGFBP1, IGFBP2, IGFBP3, IGFBP4, IGFBP5, IGFBP6, or IGFBP7, or a derivative thereof.

56. The method of any one of claims 1-53, wherein said assaying comprises detecting or quantifying at least three IGFBP1, IGFBP2, IGFBP3, IGFBP4, IGFBP5, IGFBP6, or IGFBP7, or a derivative thereof.

57. The method of any one of claims 1-53, wherein said assaying comprises detecting or quantifying at least four of IGFBP1, IGFBP2, IGFBP3, IGFBP4, IGFBP5, IGFBP6, or IGFBP7, or a derivative thereof.

58. The method of any one of claims 1-53, wherein said assaying comprises detecting or quantifying all of IGFBP1, IGFBP2, IGFBP3, IGFBP4, IGFBP5, IGFBP6, or IGFBP7, or a derivative thereof.

59. The method of any one of claims 1-58, wherein said assaying comprises detecting or quantifying leptin or a derivative thereof.

60. The method of any one of claims 1-58, wherein said assaying comprises detecting or quantifying GLP-1 and leptin, or any derivative(s) thereof, among said analyte proteins.

61. The method of any one of claims 1-60, further comprising, diagnosing, monitoring, or screening a subject for a disease or disorder based on said presence or absence of said one or more analyte proteins.

62. The method of claim 61, wherein said diagnosing, monitoring, or screening comprises identifying the presence or absence of at least a subset of said one or more analyte proteins.

63. Use of a sugar-functionalized particle for targeted detection or quantification of one or more analyte proteins selected from IGF binding protein 1 (IGFBP1), IGF binding protein 2 (IGFBP2), IGF binding protein 3 (IGFBP3), IGF binding protein 4 (IGFBP4), IGF binding protein 5 (IGFBP5), IGF binding protein 6 (IGFBP6), IGF binding protein 7 (IGFBP7), and Insulin-like growth factor binding protein acid labile subunit (IGFALS) in a biological sample.

64. Use of a sugar-functionalized particle for quantifying an amount of IGF-1 and IGF-2 in a biological sample or fraction thereof.

65. The use of any one of claim 63 or 64, wherein said sugar-functionalized particle comprises a phosphorylated sugar functionalization.

66. The use of any one of claims 63-65, wherein said sugar-functionalized particle comprises a phosphorylated monosaccharide functionalization.

67. The use of any one of claims 63-65, wherein said sugar-functionalized particle comprises a glucose 6-phosphate (G6P) functionalization.

68. Use of a polymethacrylamide or polyacrylamide coated particle for targeted detection or quantification of glucagon-like peptide 1 (GLP-1) and/or leptin, wherein said polymethacrylamide or polyacrylamide comprises an amine functional group.

69. A method of analyzing a plasma or serum sample, comprising:
(a) incubating said plasma or serum sample with a phosphorylated-sugar functionalized particle to form a protein corona; and
(b) assaying proteins in the protein corona using targeted mass spectrometry to quantify insulin-like growth factor I (IGF-1) and insulin-like growth factor II (IGF-2).

70. The method of claim 69, wherein (b) further comprises quantifying one or more analyte proteins selected from the group consisting of IGF binding protein 1 (IGFBP1), IGF binding protein 2 (IGFBP2), IGF binding protein 3 (IGFBP3), IGF binding protein 4 (IGFBP4), IGF binding protein 5 (IGFBP5), IGF binding protein 6 (IGFBP6), IGF binding protein 7 (IGFBP7), and Insulin-like growth factor binding protein acid labile subunit (IGFALS).

71. The method of claim 70, wherein at least two, at least three, at least four, at least five, at least six, at least seven, or all of said analyte proteins are quantified.

72. The method of any one of claims 70-71, wherein said particle is functionalized with a phosphorylated monosaccharide.

73. A method of analyzing a plasma or serum sample, comprising:
(a) incubating said plasma or serum sample with a polymethacrylamide or polyacrylamide coated particle to form a protein corona, wherein said polymethacrylamide or polyacrylamide comprises an amine functional group; and
(b) assaying proteins in the protein corona using targeted mass spectrometry to quantify glucagon-like peptide 1 (GLP-1) or leptin.
